SEQUENCE LISTING (29 sequences)

SEQ ID NO: 1 - protein, 20 aa, Streptococcus pyogenes
LRRDLDASREAKKQVEKALE

SEQ ID NO: 2 - protein, 20 aa, Artificial Sequence
  M protein derived from Streptococcus pyogenes
LRRDLDASREAKNQVERALE

SEQ ID NO: 3 - protein, 29 aa, Artificial Sequence
  M protein derived from Streptococcus pyogenes
KQAEDKVKASREAKKQVEKALEQLEDKVK

SEQ ID NO: 4 - protein, 28 aa, Artificial Sequence
  M protein  derived from Streptococcus pyogenes
QAEDKVKQSREAKKQVEKALKQLEDKVQ

SEQ ID NO: 5 - protein, 12 aa, Streptococcus pyogenes
SREAKKQVEKAL

SEQ ID NO: 6 - protein, 20 aa, Artificial Sequence
  Modified p145 minimal epitope fragment derived from Streptococcus pyogenes
LRRDLDASREAKNQVERALE

SEQ ID NO: 7 - protein, 27 aa, Artificial Sequence
  J8 peptide variant from Streptococcus pyogenes
SREAKKQSREAKKQVEKALKQVEKALC

SEQ ID NO: 8 - protein, 27 aa, Artificial Sequence
  J8 peptide variant from Streptococcus pyogenes
SREAKKQSREAKKQVEKALKQSREAKC

SEQ ID NO: 9 - protein, 27 aa, Artificial Sequence
  J8 peptide variant from Streptococcus pyogenes
SREAKKQVEKALKQSREAKKQVEKALC

SEQ ID NO: 10 - protein, 27 aa, Artificial Sequence
  J8 peptide variant derived from Streptococcus pyogenes
SREAKKQVEKALDASREAKKQVEKALC

SEQ ID NO: 11 - protein, 7 aa, Artificial Sequence
  GCN4 leucine zipper derived from yeast
MKQLEDK

SEQ ID NO: 12 - protein, 7 aa, Artificial Sequence
  GCN4 leucine zipper peptide derived from yeast
VKQLEDK

SEQ ID NO: 13 - protein, 20 aa, Artificial Sequence
  p145 peptide variant derived from Streptococcus pyogenes
LRRDLDAENEAKKQVEKALE

SEQ ID NO: 14 - protein, 20 aa, Artificial Sequence
  p145 peptide variant derived from Streptococcus pyogenes
LRRDLDAEDEAKKQVEKALE

SEQ ID NO: 15 - protein, 20 aa, Artificial Sequence
  p145 peptide variant derived from Streptococcus pyogenes
LRRDLDAEREAKNQVEKALE

SEQ ID NO: 16 - protein, 20 aa, Artificial Sequence
  p145 peptide variant derived from Streptococcus pyogenes
LRRDLDAEREAKKQVERALE

SEQ ID NO: 17 - protein, 20 aa, Artificial Sequence
  p145 peptide variant derived from Streptococcus pyogenes
LRRDLDAEREAKKQVEMALE

SEQ ID NO: 18 - protein, 20 aa, Artificial Sequence
  p145 peptide variant derived from Streptococcus pyogenes
LRRDLDAVNEAKKQVEKALE

SEQ ID NO: 19 - protein, 20 aa, Artificial Sequence
  p145 peptide variant derived from Streptococcus pyogenes
LRRDLDAVDEAKKQVEKALE

SEQ ID NO: 20 - protein, 20 aa, Artificial Sequence
  p145 peptide variant derived from Streptococcus pyogenes
LRRDLDAVDEAKKQVEKALE

SEQ ID NO: 21 - protein, 20 aa, Artificial Sequence
  p145 peptide variant derived from Streptococcus pyogenes
LRRDLDAVREAKKQVERALE

SEQ ID NO: 22 - protein, 20 aa, Artificial Sequence
  p145 peptide variant derived from Streptococcus pyogenes
LRRDLDAVREAKKQVEMALE

SEQ ID NO: 23 - protein, 20 aa, Artificial Sequence
  p145 peptide variant derived from Streptococcus pyogenes
LRRDLDASNEAKNQVEKALE

SEQ ID NO: 24 - protein, 20 aa, Artificial Sequence
  p145 peptide variant derived from Streptococcus pyogenes
LRRDLDASNEAKKQVERALE

SEQ ID NO: 25 - protein, 20 aa, Artificial Sequence
  p145 peptide variant derived from Streptococcus pyogenes
LRRDLDASNEAKKQVEMALE

SEQ ID NO: 26 - protein, 20 aa, Artificial Sequence
  p145 peptide variant derived from Streptococcus pyogenes
LRRDLDASDEAKNQVEKALE

SEQ ID NO: 27 - protein, 20 aa, Artificial Sequence
  p145 peptide variant derived from Streptococcus pyogenes
LRRDLDASDEAKKQVERALE

SEQ ID NO: 28 - protein, 20 aa, Artificial Sequence
  p145 peptide variant derived from Streptococcus pyogenes
LRRDLDASDEAKKQVEMALE

SEQ ID NO: 29 - protein, 20 aa, Artificial Sequence
  p145 peptide variant derived from Streptococcus pyogenes
LRRDLDASREAKNQVEMALE

(19) United States
(12) Patent Application Publication
(10) Pub. No.: US 2021/0292398 A1
(43) Pub. Date: Sep. 23, 2021

(54) STREPTOCOCCAL TOXIC SHOCK SYNDROME

(71) Applicant: GRIFFITH UNIVERSITY, Nathan (AU)

(72) Inventors: Michael GOOD, Nathan (AU); Manisha PANDEY, Nathan (AU)

(73) Assignee: Griffith University, Nathan (AU)

(21) Appl. No.: 17/055,294

(22) PCT Filed: May 16, 2019
(86) PCT No.: PCT/AU2019/050469

§ 371 Date: Nov. 13, 2020

(30) Foreign Application Priority Data
May 16, 2018 (AU) 2018901709
Nov. 16, 2018 (AU) 2018904377

(51) Int. Cl. C07K 16/12; A61P 31/04; C07K 14/315

(52) U.S. Cl. CPC C07K 16/1275; A61P 31/04; A61K 2039/505; C07K 2317/14; C07K 14/315

(57) ABSTRACT

Provided herein are methods of immunizing against, treating or preventing streptococcal toxic shock syndrome in a subject, by administration of a group A Streptococcus M protein, inclusive of fragments, variants or derivatives thereof, or an antibody that binds, or is raised against the M protein and optionally a group A Streptococcus superantigen protein, inclusive of fragments, variants or derivatives thereof, or an antibody or antibody fragment that binds, or is raised against, the superantigen protein.

### CROSS-REFERENCE TO RELATED APPLICATIONS

This application is a United States National Phase Application filed under 35 U.S.C. § 371 from International Patent Application No. PCT/AU2019/050469, filed on May 16, 2019, which claims priority from Australian Patent Application Nos. 2018904377, filed on Nov. 16, 2018 and 2018901709, filed on May 16, 2018. The contents and disclosures of each of these applications are incorporated by reference herein in their entireties.

### SEQUENCE LISTING

The instant application contains a Sequence Listing which has been submitted electronically as a text file in ASCII format and is hereby incorporated by reference in its entirety. Said text file, created on Nov. 12, 2020, is named 000336-0001-301-SL.txt and is 9665 bytes in size.

### FIELD

THIS INVENTION relates to prevention and treatment of diseases caused by group A streptococci. More particularly, this invention relates to an antibodies or antibody fragments for treating or preventing group A Streptococcus-associated toxic shock syndrome.

### BACKGROUND

Infections with group A Streptococcus (Streptococcus pyogenes, GAS) are highly prevalent in all sectors of society with estimates of over 600 million incident cases of streptococcal pharyngitis and over 160 million prevalent cases of streptococcal pyoderma. The vast majority of cases are benign and can be treated successfully with antibiotics and basic health care. However, streptococcal disease can progress beyond the throat and skin, giving rise to invasive GAS (‘iGAS’) disease, including streptococcal toxic shock syndrome (STSS). Furthermore, untreated infections can give rise to post streptococcal sequelae including rheumatic heart disease and glomerulonephritis. iGAS disease and post streptococcal sequelae are particularly prevalent amongst Aboriginal and Torres Strait Islander populations, and amongst socially disadvantaged populations throughout the world.

Globally, these conditions are responsible for the loss of over 500,000 lives per year. Conservative estimates now place GAS as the fourth most common cause of infection-related mortality globally (after HIV, tuberculosis and Streptococcus pneumoniae). These numbers are considered to be the ‘tip of the iceberg’ with there being a current epidemic of iGAS disease in both developed and underdeveloped nations.

STSS is caused primarily by superantigen toxins that bind non-specifically to human MHC II molecules (outside the peptide binding groove) and T-cell receptor variable chains, resulting in polyclonal T-cell activation often with >20% of CD4+ T-cells being activated. This results in a Th1 cytokine storm which is the proposed causal link responsible for hypotension and multi-organ failure, (which includes the liver, kidney, coagulation system and respiratory system).

In mouse models it has been shown that T cells are required for superantigen-mediated mortality. In a model using a staphylococcal superantigen (SEB), it was also shown that anti-TNF pre-treatment could block the lethality of toxic shock [2]. STSS has a very high mortality, which can exceed 50%, even in high income countries. This condition can occur after any streptococcal infection but most commonly occurs after infections of the skin. It is usually associated with necrotising fasciitis, myositis or deep bruising. Chickenpox, cellulitis and direct skin puncture can be significant co-factors.

Superantigens (SAgs) are low molecular weight exo-proteins that are secreted by all pathogenic GAS and Staphylococcus aureus strains. There are 11 serologically distinct superantigens in GAS. Nine of the 11 are located on genes present in bacteriophages. They can activate primary T cells and do not require antigen processing. Superantigens demonstrate high affinity binding to the human MHC II β chain and relatively low affinity binding to TCR β chains. The affinity of superantigens for mouse MHC is several orders of magnitude lower than for human MHC [3] and as such, normal mice are not suitable models for studying superantigen-mediated disease. Of the 11 superantigens that can be present in GAS, most cases of STTS are caused by one or other of Streptococcal pyrogenic exotoxin (Spe) A or SpeC [4].

Efforts to develop vaccines to prevent STSS are limited. One group has developed toxoids to SpeA and SpeC and shown that vaccination of rabbits can lead to antibodies that neutralize the toxin and protect rabbits from native toxin administered via a mini-osmotic pump. The rabbits were not exposed to a streptococcal infection [4, 5]. This vaccine approach suffers from the need to vaccinate with multiple toxoids to protect against only one aspect of streptococcal disease.

HLA transgenic mice have been used as a model to develop a candidate vaccine using defined non-toxic fragments of superantigens from S. aureus [3]. These mice were not challenged with the organism, but with recombinant superantigen.

Passive immunotherapy has been examined as a means to treat STSS. Intravenous immunoglobulin (IVIG) has been shown to significantly reduce the case fatality of STSS [6]. This study used historical controls but in a more recent Swedish study of 67 patients with prospective controls, the mortality was 22 from 44 patients treated with antibiotics alone (50%) vs 3 from 23 (13%) in the group treated with IVIG plus antibiotics (P<0.01) [7]. However, it has been estimated that superantigen antibody titres of >40 in the IVIG are required for clinical benefit. This is approximately the amount of specific antibody that is found in IVIG and as such multiple doses of IVIG are recommended. The high costs of IVIG, batch to batch variation [8] and difficulties in supply underscore the need for alternative adjunctive therapies.

### SUMMARY

Surprisingly, the present inventors have discovered that antibodies or antibody fragments that bind a group A Streptococcus M protein fragment or variant thereof with or without antibodies or antibody fragments that bind a group A Streptococcus superantigen fragment or variant thereof are surprisingly efficacious against a group A Streptococcus-associated disease disorder or condition such as streptococcal toxic shock syndrome.

In a broad form, the invention therefore relates to use of antibodies or antibody fragments that bind a group A Streptococcus M protein, fragment, variant or derivative thereof and optionally an antibody or antibody fragment that binds a group A Streptococcus superantigen protein, fragment, variant or derivative thereof to passively immunize against, treat or prevent a group A Streptococcus-associated disease disorder or condition such as invasive GAS (iGAS) disease inclusive of streptococcal toxic shock syndrome (STSS).

In another broad form, the invention relates to the use of a group A Streptococcus M protein fragment, variant or derivative thereof and optionally a group A Streptococcus superantigen protein, fragment, variant or derivative thereof to vaccinate or immunize against, treat or prevent a group A Streptococcus-associated disease disorder or condition such as invasive GAS (iGAS) disease inclusive of streptococcal toxic shock syndrome (STSS).

An aspect of the invention provides a method of passively immunizing a mammal against streptococcal toxic shock syndrome, said method including the step of administering to the mammal: an antibody or antibody fragment that binds, or is raised against, a group A Streptococcus M protein, fragment, variant or derivative thereof, to thereby passively immunize the mammal against streptococcal toxic shock syndrome in the mammal.

In one particular embodiment of the aforementioned aspects, the method further includes the step of administering an antibody or antibody fragment that binds, or is raised against, a group A Streptococcus superantigen to the mammal.

Another aspect of the invention provides a method of treating or preventing streptococcal toxic shock syndrome in a mammal, said method including the step of administering to the mammal: a group A Streptococcus M protein, fragment, variant or derivative thereof and/or an antibody or antibody fragment that binds, or is raised against, a group A Streptococcus M protein, fragment, variant or derivative thereof to thereby treat or prevent streptococcal toxic shock syndrome in the mammal.

In one particular embodiment of the aforementioned aspects, the method further includes the step of administering a group A Streptococcus superantigen protein, fragment, variant or derivative thereof and/or an antibody or antibody fragment that binds, or is raised against, a group A Streptococcus superantigen protein, fragment, variant or derivative thereof to the mammal.

A further aspect of the invention provides a composition suitable for administration to a mammal, said composition comprising: an antibody or antibody fragment that binds, or is raised against, a group A Streptococcus M protein, fragment, variant or derivative thereof.

In one embodiment of the present aspect, the composition further comprises an antibody or antibody fragment that binds, or is raised against, a group A Streptococcus superantigen protein, fragment, variant or derivative thereof.

For the aforementioned aspects, the antibody or antibody fragment is suitably a monoclonal antibody or antibody fragment. In one particular embodiment of the aforementioned aspects, the monoclonal antibody or antibody fragment is a recombinant humanized monoclonal antibody or fragment thereof.

In a related aspect, the invention resides in a composition suitable for administration to a mammal, said composition comprising: a group A Streptococcus M protein, fragment, variant or derivative thereof and a group A Streptococcus superantigen protein, fragment, variant or derivative thereof.

A further related aspect of the invention provides a monoclonal antibody or fragment thereof which binds, or is raised against, a group A Streptococcus M protein, fragment, variant or derivative thereof; and/or an antibody or antibody fragment that binds, or is raised against, a group A Streptococcus superantigen protein, fragment, variant or derivative thereof.

Preferably, the monoclonal antibody or fragment is a recombinant humanized monoclonal antibody or fragment thereof.

This aspect also provides an isolated nucleic acid encoding the recombinant humanized monoclonal antibody or fragment thereof, a genetic construct comprising the isolated nucleic acid and/or a host cell comprising the genetic construct.

In a particular embodiment of the aforementioned aspects, the M protein fragment is or comprises a conserved region of the M protein. In one embodiment, the M protein fragment is, comprises, or is contained within a p145 peptide.

In one particular embodiment, the M protein fragment is, is contained within, or comprises, a J8 peptide, fragment, variant or derivative thereof.

In another particular embodiment, the fragment is, is contained within, or comprises, a p17 peptide, fragment, variant or derivative thereof.

In another particular embodiment of the aforementioned aspects, the superantigen is streptococcal pyrogenic exotoxin (Spe) A or SpeC.

Suitably, according to the aforementioned aspects the mammal is a human.

As used herein, the indefinite articles ‘a’ and ‘an’ are used here to refer to or encompass singular or plural elements or features and should not be taken as meaning or defining “one” or a “single” element or feature.

Unless the context requires otherwise, the terms “comprise”, “comprises” and “comprising”, or similar terms are intended to mean a non-exclusive inclusion, such that a recited list of elements or features does not include those stated or listed elements solely, but may include other elements or features that are not listed or stated.

By “consisting essentially of” in the context of an amino acid sequence is meant the recited amino acid sequence together with an additional one, two or three amino acids at the N- or C-terminus.

### BRIEF DESCRIPTION OF THE DRAWINGS

FIG. 1: (A-B) Infectivity of GAS SN1 in HLA-B6 mice. Naïve HLA-B6 and B6 mice (n=10/group) were infected with GAS SN1 via skin. On day 6-post infection mice were culled and skin bacterial burdens were assessed (A). The presence of systemic infection was assessed by plating blood samples at day 3, 4, 5 and 6 post infection (B) ***p<0.001. (C-D). Western blot analysis of serum from SN1 infected mice. Serum samples collected from SN1 infected BALB/c (C) and SN1 and NS33 (a group C Streptococcus that does not express superantigens) infected HLA-B6 and B6 mice (D) were analysed to detect the presence of SpeC in their serum. The samples were run on 4-15% SDS-PAGE gel. Following protein transfer from the gel, the membrane was probed with primary antibody, Rabbit anti-SpeC IgG, followed by detection with Sheep anti-rabbit IgG-AP and developed using BCIP/NBT substrate. The band at ˜26 KDa in serum sample from SN1 infected mice corresponds to rSpeC in the positive control sample.

FIG. 2: (A) Mitogenic activity of SpeC in a murine model. Splenocyte proliferation in response to SN1 SpeC. Splenocytes from HLA-B6 and B6 mice were stimulated in vitro with either sterile filtered SpeC-containing serum from SN1 GAS-infected mice or with rSpeC. As controls, sterile filtered serum from mice infected with superantigen negative GAS strain (NS33) and ConA were also included. Proliferation of splenocytes was assessed after 72 h and data are represented as stimulation indices (SI). The specificity of response was confirmed by addition of anti rSpeC antibodies, which inhibited the proliferation of splenocytes in response to serum and rSpeC. (B-C). Cytokine profiles following splenocyte proliferation. Cytokine responses in splenocytes from HLA-B6 and B6 mice were measured at 72 h post incubation with various stimulants. Concentrations of TNF (B) and IFN-γ (C) in the culture supernatants were measured using a CBA kit. The specificity of response was confirmed by addition of anti-rSpeC antibodies. One-way ANOVA with Tukey's post-hoc method was utilised to calculate significance between various groups. *p<0.05 and **p<0.01. SI was defined as counts per minute in the presence of antigen/counts per minute in the absence of antigen.

FIG. 3. (A). Protective efficacy of J8-DT against GAS SN1 infection. HLA-B6 mice were vaccinated with J8-DT or PBS on day 0, 21 and 28. Two weeks post-immunisation mice were infected with GAS SN1 via the skin. On day 6 post-infection mice were culled and bacterial burden in skin (CFU/lesion), blood (cfu/mL) and spleen (CFU/spleen) are shown. (B). Western blot analysis to detect toxin in serum. Pooled serum samples from vaccinated and control cohorts collected at day 6 post SN1 infection, were run on 4-15% SDS-PAGE gels. Following protein transfer from the gel, the membrane was probed with Rabbit anti-SpeC IgG followed by detection with Sheep anti-rabbit IgG-AP and developed using BCIP/NBT substrate. The band at 26 KDa in serum sample from PBS mice corresponds to rSpeC in the positive control sample. (C-D). Assessment of proliferation induced by serum from vaccinated infected mice. PBMCs from 2 different individuals were stimulated with pre-optimized concentration of serum from SN1 infected-vaccinated (J8-DT+SN1) or un-vaccinated control (PBS+SN1) mice. PHA and rSpeC were used as controls for stimulation. The specificity of response was assessed by addition of various amounts of rSpeC antisera. PBMC in the presence of naïve sera was used as a control for specificity of neutralization. Proliferation was measured by [³H]thymidine uptake after 72 h. Data are Mean±SEM of 3 replicates in each experiment with experiments repeated twice. Representative data from two individuals are shown. One-way ANOVA with Tukey's post-hoc method was utilised to calculate significance. *p<0.05, **p<0.01 and ***p<0.001.

FIG. 4. (A-C). Neutralization of rSpeC by rSpeC antisera. PBMCs from 3 different individuals were stimulated with different concentrations of rSpeC in the presence of various amounts of rSpeC antiserum or no serum. PHA was used as control. Proliferation was measured by [³H] thymidine uptake after 72 h. Data are Mean±SEM of 3 replicates in each experiment with experiments repeated twice. Stimulation index (SI) was defined as counts per minute in the presence of antigen/counts per minute in the absence of antigen. One-way ANOVA with Tukey's post-hoc method was utilised to calculate significance. *p<0.05 and ***p<0.001.

FIG. 5. (A) Challenge study with GAS incubated with J8-DT antisera. GAS 2031(emm1) strain was incubated with rotation for 1 h at 4° C. with 1:50 dilution of J8-DT antiserum. Following washes the bacterial inocula was injected intraperitoneally into SCID mice. After 48 h, mice were culled and blood harvested. The bacterial burdens in individual mice are shown. (B) In vivo neutralisation of SpeC by rSpeC antisera. SN1 infected BALB/c mice were administered anti-rSpeC or naïve sera intraperitoneally on day 5 post-infection. To assess SpeC neutralisation in vivo, sera samples were collected prior to (0 h) and then at 6 and 24 h post antisera administration. The presence of SpeC in mouse sera at various time-points are shown. (C) Effect of rSpeC antisera treatment on skin bacterial burden. SN1 infected BALB/c mice were administered anti-rSpeC or naïve sera intraperitoneally on day 5 post-infection. At 24 h post-treatment, the mice were culled and bacterial burdens assessed. Bacterial burdens in skin for treated and untreated mice are shown. Statistical analysis was performed using non-parametric, unpaired Mann-Whitney U-test to compare the two groups. **p<0.01.

FIG. 6. (A-B) Virulence of human isolates in murine skin infection model. Cohorts of BALB/c mice were infected with GAS SN1 or GAS NS33 strain via the skin route of infection. Post day 3, 6 or 9 of challenge, the mice were culled and skin biopsy (A) and spleen (B) samples were collected to determine the bacterial burden. The results are shown as box and whisker plot where the line in the box is indicating the median, the box extremities indicating the upper and lower quartiles and the whiskers showing minimum to the maximum values. (C) SpeC detection in individual mouse serum sample from day 6 collection. Serum sample from each individual mouse on day 6 following SN1/NS33 infection were also assessed for presence of SpeC as described. A representative image is shown. The * indicates the mice that had positive spleen culture. Statistical analysis was performed using non-parametric, unpaired Mann-Whitney U-test to compare the two groups at each time point. **p<0.01 and ***p<0.001.

FIG. 7. (A) Mitogenic activity of SpeC in a murine model. Splenocyte proliferation in response to SN1 SpeC. Splenocytes from HLA-B6 and B6 mice were stimulated in vitro with either sterile filtered SpeC-containing serum from SN1 GAS-infected mice or with rSpeC. As controls, sterile filtered serum from mice infected with superantigen negative GAS strain (NS33) and ConA were also included. Proliferation of splenocytes was assessed after 72 h and data are represented as stimulation indices (SI). The specificity of response was confirmed by addition of anti rSpeC antibodies, which inhibited the proliferation of splenocytes in response to serum and rSpeC. (B-C). Cytokine profiles following splenocyte proliferation. Cytokine responses in splenocytes from HLA-B6 and B6 mice were measured at 72 h post incubation with various stimulants. Concentrations of TNF (B) and IFN-γ (C) in the culture supernatants were measured using a CBA kit (BD Biosciences). The specificity of response was confirmed by addition of anti-rSpeC antibodies. One-way ANOVA with Tukey's post-hoc method was utilised to calculate significance between various groups.*p<0.05 and **p<0.01. SI was defined as counts per minute in the presence of antigen/counts per minute in the absence of antigen. (D-F). Proliferation of human PBMC in response to stimulation with serum from GAS SN1 or GAS NS33 infected mice. PBMC from three different individuals were cultured in the presence of serum collected at various time-points following infection with GAS SN1 or GAS NS33. Proliferation was measured by [³H]thymidine uptake after 72 h. Data are Mean±SEM of 3 replicates in each experiment with experiments repeated twice.

FIG. 8. (A-B). In vivo infectivity of GAS SN1 in HLA-B6 mice. Naïve HLA-B6 and B6 mice (n=10/group) were infected with GAS SN1 via intraperitoneal route of GAS infection. Mice received either 10⁶, 10⁷ or 10⁸ CFU of SN1. At 24 h post-infection mice were scored for clinical symptoms to assess severity of disease. The clinical scores for both HLA-B6 and B6 mice are shown. (B) Following scoring mice were culled and bacterial burden in blood and spleens assessed. The results are shown as box and whisker plot where the line in the box is indicating the median, the box extremities indicating the upper and lower quartiles and the whiskers showing minimum to the maximum values. One-way ANOVA with Tukey's post-hoc method was utilised to calculate significance between the control and test groups. (C) Western blot analysis of serum from SN1 infected HLA-B6 and B6 mice. Serum samples collected from SN1 infected mice were analysed to detect the toxin in their serum. The samples were run on 4-15% SDS-PAGE gel. Following protein transfer from the gel, the membrane was probed with primary antibody, Rabbit anti-SpeC IgG, followed by detection with Sheep anti-rabbit IgG-AP and developed using BCIP/NBT substrate. rSpeC protein was also run as a positive control. (D-F) Serum cytokine profile of HLA-B6 mice following intra-peritoneal infection with SN1. The mice infected with SN1 were culled at 24 h post-infection The blood cytokine levels were measured in blood samples collected from the cohort that received the highest dose (1×10⁸ CFU) of SN1 at using a CBA kit. TNF, IFN-T and IL-2 responses are shown. One-way ANOVA with Tukey's post-hoc method was utilised to calculate significance between various groups.*p<0.05 and **p<0.01. SI was defined as counts per minute in the presence of antigen/counts per minute in the absence of antigen.

FIG. 9. (A-B). Infectivity of GAS SN1 in HLA-B6 mice. Naïve HLA-B6 and B6 mice (n=10/group) were infected with GAS SN1 or GAS NS33 via skin. On day 6-post infection mice were culled and skin bacterial burdens were assessed (A). The presence of systemic infection was assessed by plating blood samples at day 3, 4, 5 and 6 post infection (B). The results are shown as box and whisker plot where the line in the box is indicating the median, the box extremities indicating the upper and lower quartiles and the whiskers showing minimum to the maximum values. (C) Western blot analysis of serum from SN1 or NS33 infected mice. Serum samples collected from SN1 or NS33 infected HLA-B6 and B6 mice were analysed to detect the presence of SpeC in their serum. The samples were run on 4-15% SDS-PAGE gel. Following protein transfer from the gel, the membrane was probed with primary antibody, Rabbit anti-SpeC IgG, followed by detection with Sheep anti-rabbit IgG-AP and developed using BCIP/NBT substrate. The band at 26 KDa in serum sample from SN1 infected mice corresponds to rSpeC in the positive control sample. (D-F). Cytokine responses in the serum of HLA-B6 and B6 mice following skin infection. Cytokine responses in the serum of HLA-B6 and B6 mice were measured at day 6 post infection with SN1 or NS33. Concentration of TNF (C), IFN-T (D) and IL-2 were measured using a CBA kit. One-way ANOVA with Tukey's post-hoc method was utilised to calculate significance between various groups. ***p<0.001.

FIG. 10. (A). Protective efficacy of J8-DT against GAS SN1 infection. HLA-B6 mice were vaccinated with J8-DT or PBS on day 0, 21 and 28. Two weeks post-immunization mice were infected with GAS SN1 via the skin. On day 6 post-infection mice were culled and bacterial burden in skin (CFU/lesion), blood (cfu/mL) and spleen (CFU/spleen) are shown. (B). Western blot analysis to detect toxin in serum. Pooled serum samples from vaccinated and control cohorts collected at day 6 post SN1 infection, were run on 4-15% SDS-PAGE gels. Following protein transfer from the gel, the membrane was probed with Rabbit anti-SpeC IgG followed by detection with Sheep anti-rabbit IgG-AP and developed using BCIP/NBT substrate. The band at 26 KDa in serum sample from PBS mice corresponds to rSpeC in the positive control sample. (C-D). Cytokine responses in the serum of HLA-B6 mice following skin infection. Cytokine responses in the serum of vaccinated and control HLA-B6 mice were measured at day 6 post infection with SN1. Concentration of IL-4 and IL-10 (C) and TNF and IFN-T (D) were measured using a CBA kit. One-way ANOVA with Tukey's post-hoc method was utilised to calculate significance between various groups. ***p<0.001. (E-G). Assessment of proliferation induced by serum from vaccinated/control-infected mice. PBMCs from 3 different individuals were stimulated with pre-optimized concentration of serum from vaccinated-SN1 infected (J8-DT+SN1) or un-vaccinated-SN1 infected (PBS+SN1) mice. PHA and rSpeC were used as controls for stimulation. The specificity of response was assessed by addition of various amounts of rSpeC antisera. PBMC in the presence of naïve sera was used as a control for specificity of neutralization. Proliferation was measured by [³H] thymidine uptake after 72 h. Data are Mean±SEM of 3 replicates in each experiment with experiments repeated twice. Representative data from two individuals are shown. One-way ANOVA with Tukey's post-hoc method was utilised to calculate significance. *p<0.05, **p<0.01 and ***p<0.001.

FIG. 11. Cytokine response of PBMC following stimulation with vaccinated and control sera. PBMC from three different individuals were stimulated with pre-optimized concentration of serum from vaccinated-SN1 infected or control-SN1 infected mice. Optimal concentrations of rSpeC and PHA were used as a positive control for stimulation. The inhibitory effect of rSpeC antisera was assessed by adding a pre-optimized amount (20 μL) of rSpeC antisera to selected wells containing vaccinated-SN1 infected or control-SN1 infected sera or rSpeC. Media alone wells were used as negative controls. Cytokine responses were measured using CBA kit after 72 h of in vitro culture. Data are Mean±SEM of 3 replicates in each experiment with experiments repeated twice. Statistical analysis was performed using non-parametric, unpaired Mann-Whitney U-test to compare the two groups. *p<0.05, **p<0.01 and ***p<0.001.

FIG. 12. (A) In vivo neutralisation of SpeC by rSpeC antisera. HLA-B6 mice were infected with GAS SN1 via skin. On day 5 post-infection mice were administered anti-rSpeC or naïve sera intraperitoneally. To assess SpeC neutralisation in vivo, sera samples were collected prior to (0 h) and then at 6 and 24 h post antisera administration. The presence of SpeC in treated and untreated HLA-B6 mice sera at various time-points are shown. (B) Therapeutic potential of rSpeC antisera. To assess the therapeutic potential of rSpeC antisera, designated number of mice were culled at 6 and 24 h post serum administration. Bacterial burden in skin and blood of treated and untreated mice are shown. The results are shown as box and whisker plot where the line in the box is indicating the median, the box extremities indicating the upper and lower quartiles and the whiskers showing minimum to the maximum values. NS p>0.05.

FIG. 13. Therapeutic potential of combination immunotherapy (A) Time-line of infection and treatment protocol (B) Four cohorts of HLA-B6 mice (n=3-5/group) were infected intraperitoneally with a pre-optimised dose of GAS SN1. Eighteen hour post-infection mice were scored for clinical symptoms and intravenously administered 200 μL of either anti-J8-DT, anti-rSpeC, a combination of anti-J8-DT and anti-rSpeC or naïve sera. At 24 h post treatment (42 h post-infection) mice were assessed for clinical scores and then culled. Blood and spleen samples were harvested, processed and plated for quantification of bacteria. The bacterial burdens in blood and spleen of mice are shown. (C) All mice were scored for clinical symptoms before and after treatment to assess disease severity. The clinical scores for all cohorts before (0 h) and after (24 h) antisera treatment are shown. (D-G) To assess SpeC neutralisation in-vivo, sera samples from all cohorts were collected prior to (0 h) and then at 24 h post antisera administration. The presence of SpeC in HLA-B6 mice treated with J8-DT antiserum (D), rSpeC antiserum (E) J8-DT+rSpeC antiserum (F) or PBS antiserum (G) sera before and after treatment are shown. Mann-Whitney test was performed to compare each group with the control PBS treated group. *p<0.05, **p<0.01, ***p<0.001 and NS p>0.05.

FIG. 14. Splenocyte proliferation and inhibition in response to StrepA antigens and various antisera. (A) Assessment of proliferation in response to SN1 infected sera and its inhibition by antisera. Splenocyte proliferation was assessed in response to SpeC-containing serum from SN1 GAS-infected mice in the presence or absence of J8-DT, rSpeC, J8-DT+rSpeC or PBS antisera. (B) Splenocytes stimulated with rSpeC, rM1 or rSpeC+rM1 were also included as controls. As blocking agent J8-DT, rSpeC or J8-DT+rSpeC antisera were used. Proliferation of splenocytes was assessed after 72 h and data are represented as stimulation indices (SI). **p<0.01, ***p<0.001 and NS p>0.05.

FIG. 15. The genomic DNA was extracted from overnight stationary phase cultures using the GenElute bacterial gDNA extraction kit from Sigma. The gDNA was qualified using the Nanodrop1000 and then 2 ug of gDNA used for amplification of the superantigens. Gels were then run as per the image legend.

FIG. 16. In vitro growth of GAS human isolates in murine blood. GAS isolates were grown O/N in THB with 1% neopeptone. Each isolate was serially diluted up to 10⁻⁶ and incubated with fresh heparinized murine blood in a ratio of 1:3. Bacterial growth in murine blood was measured after 3 h incubation at 37° C. and compared with the CFU counts in the starting culture. Isolates showing >20 fold increase in CFU were defined as isolates with higher potential to cause systemic streptococcal infections in a murine model. The data shown is the mean SEM for each isolate.

FIG. 17. Proliferative response of human PBMC in response to stimulation with serum from GAS NS1 or GAS NS33 infected mice. PBMC from three different individuals were stimulated with different volumes of serum collected from mice infected with GAS SN1 or GAS NS33. PHA was used as control. Proliferation was measured by [³H] thymidine uptake after 72 h. Data are Mean±SEM of 3 replicates in each experiment with experiments repeated twice. One-way ANOVA with Tukey's post-hoc method was utilised to calculate significance between various groups. *p<0.05. **p<0.01 and ***p<0.001.

### DETAILED DESCRIPTION

The present invention is at least partly predicated on the discovery that antibodies or antibody fragments that bind a group A Streptococcus (GAS) M protein, fragment, variant or derivative thereof with or without an antibody or antibody fragment that binds a group A Streptococcus superantigen protein, fragment, variant or derivative thereof are surprisingly efficacious against a group A Streptococcus-associated disease disorder or condition such as such as invasive GAS disease inclusive of streptococcal toxic shock syndrome (STSS).

In a broad form, the invention therefore relates to the use of antibodies or antibody fragments that bind a group A Streptococcus M protein fragment or variant thereof and optionally an antibody or antibody fragment that binds a group A Streptococcus superantigen protein, fragment or variant thereof to passively immunize against, treat or prevent a group A Streptococcus-associated disease disorder or condition, such as invasive GAS disease and inclusive of streptococcal toxic shock syndrome (STSS).

In another broad form, the invention relates to the use of a group A Streptococcus M protein fragment, variant or derivative thereof and optionally a group A Streptococcus superantigen protein, fragment, variant or derivative thereof to vaccinate or immunize against, treat or prevent a group A Streptococcus-associated disease disorder or condition such as invasive GAS (iGAS) disease and inclusive of streptococcal toxic shock syndrome (STSS).

As used herein the terms “group A Streptococcus”, “Group A Streptococci”, “Group A Streptococcal”, “Group A Strep” and the abbreviation “GAS” refer to streptococcal bacteria of Lancefield serogroup A which are gram positive β-hemolytic bacteria of the species Streptococcus pyogenes. An important virulence factor of GAS is M protein, which is strongly anti-phagocytic and binds to serum factor H, destroying C3-convertase and preventing opsonization by C3b. These also include virulent “mutants” such as CovR/S or CovRS mutants such as described in Graham et al., 2002, PNAS USA 99 13855, although without limitation thereto.

Diseases, disorders and conditions caused by group A streptococci include cellulitis, erysipelas, impetigo, scarlet fever, throat infections such as acute pharyngitis (“strep throat”), bacteremia, invasive GAS diseases such as streptococcal toxic shock syndrome (STSS), necrotizing fasciitis, acute rheumatic fever and acute glomerulonephritis, although without limitation thereto. In a particular embodiment, the disease or condition is or comprises streptococcal toxic shock syndrome (STSS).

By “protein” is meant an amino acid polymer. The amino acids may be natural or non-natural amino acids, D- or L-amino acids as are well understood in the art.

The term “protein” includes and encompasses “peptide”, which is typically used to describe a protein having no more than fifty (50) amino acids and “polypeptide”, which is typically used to describe a protein having more than fifty (50) amino acids.

A “fragment” is a segment, domain, portion or region of a protein (such as M protein, p145, p17, J8 or J14 or a superantigen or an antibody raised against or directed thereto), which constitutes less than 100% of the amino acid sequence of the protein. It will be appreciated that the fragment may be a single fragment or may be repeated alone or with other fragments.

In general, fragments may comprise, consist essentially of or consist of up to 5, 6, 7, 8, 9, 10, 12, 15, 20, 25, 30, 40, 50, 60, 70, 80, 90, 100, 150, 200, 250, 300, 350, 400, 450, 500, 550, 600, 650, 700, 750, 800, 850, 900, 950, 100, 1050, 1100, 1150, 1200, 1250, 1300, 1350, 1400, 1450, 1500, 1550 or 1600 amino acids of the full length protein.

Suitably, the fragment is “immunogenic”, by which is meant the fragment can elicit an antibody response upon administration to a mammal.

As generally used herein an “antibody” is, or is derived from, a protein product of the immunoglobulin gene complex, inclusive of isotypes such as IgG, IgM, IgD, IgA and IgE and subtypes such as IgG₁, IgG₂ₐ etc, although without limitation thereto. Antibodies and antibody fragments may be polyclonal or monoclonal, native or recombinant. Antibody fragments include Fc, Fab or F(ab)2 fragments and/or may comprise single chain Fv antibodies (scFvs). Such scFvs may be prepared, for example, in accordance with the methods described respectively in U.S. Pat. No. 5,091,513, European Patent No 239,400 or the article by Winter & Milstein, 1991, Nature 349:293. Antibodies may also include multivalent recombinant antibody fragments, such as diabodies, triabodies and/or tetrabodies, comprising a plurality of scFvs, as well as dimerisation-activated demibodies (e.g. WO/2007/062466). By way of example, such antibodies may be prepared in accordance with the methods described in Holliger et al., 1993 Proc Natl Acad Sci USA 90 6444; or in Kipriyanov, 2009 Methods Mol Biol 562 177. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988.

Methods of producing polyclonal antibodies are well known to those skilled in the art. Exemplary protocols which may be used are described for example in Coligan et al., CURRENT PROTOCOLS IN IMMUNOLOGY, supra, and in Harlow & Lane, 1988, supra. In a particular embodiment, polyclonal antibodies may be obtained or purified from human sera from individuals exposed to, or infected by, Group A strep. Alternatively, polyclonal antibodies may be raised against purified, chemical synthetic or recombinant M protein, superantigens, or an immunogenic fragment or variant thereof, in production species such as horses and then subsequently purified prior to administration.

Monoclonal antibodies may be produced using the standard method as for example, originally described in an article by Köhler & Milstein, 1975, Nature 256, 495, or by more recent modifications thereof as for example, described in Coligan et al., CURRENT PROTOCOLS IN IMMUNOLOGY, supra by immortalizing spleen or other antibody producing cells derived from a production species which has been inoculated with one or more of the isolated proteins, fragments, variants or derivatives of the invention. The monoclonal antibody or fragment thereof may be in recombinant form. This may be particularly advantageous for “humanizing” the monoclonal antibody or fragment if the monoclonal antibody is initially produced by spleen cells of a non-human mammal.

In one embodiment, the antibody or antibody fragment binds and/or is raised against an M protein, fragment or variant thereof.

As used herein an “M protein fragment” is any fragment of a GAS M protein that is immunogenic and/or is capable of being bound by an antibody or antibody fragment. Typically, the fragment is, comprises, or is contained within an amino acid sequence of a C-repeat region of a GAS M protein, or a fragment thereof. Non-limiting examples include p145, which is a 20mer with the amino acid sequence LRRDLDASREAKKQVEKALE (SEQ ID NO:1). A minimal p145 epitope sequence is SREAKKQVEKAL (SEQ ID NO:5).

In particular embodiments, the M protein fragment is or comprises the minimal p145 epitope of SEQ ID NO: 5 or a variant or derivative thereof.

In this regard, fragments of the p145 amino acid sequence may be present in p17, J14 or J8 peptides. Accordingly, in particular embodiments, the M protein fragment, variant or derivative thereof consists, consists essentially of or comprises a p17 peptide, a J14 peptide or a J8 peptide.

In work performed prior to the present invention, certain modifications to p145 peptide can substantially improve immunogenicity against group A streptococci. In one embodiment, a p17 peptide is a modified p145 peptide that comprises an N residue corresponding to residue 13 of SEQ ID NO:1 and an R amino acid at residue 17 of SEQ ID NO:1.

Preferably, p17 comprises a modified p145 minimal epitope that comprises an N residue corresponding to residue 6 of SEQ ID NO:5 and an R amino acid at residue 10 of SEQ ID NO:1.

In one embodiment, a p17 peptide comprises the amino acid sequence LRRDLDASREAKNQVERALE (SEQ ID NO:2).

In one embodiment, a p17 peptide comprises a modified p145 minimal epitope fragment that comprises the amino acid sequence SREAKNQVERAL (SEQ ID NO:6).

Additional p145 peptide variants are outlined in PCT/AU2018/050893, which is incorporated by reference herein. Exemplary p145 variants are provided below:

p145
(SEQ ID NO: 1)
LRRDLDA SREAKKQVEKAL E
p*l.
(SEQ ID NO: 13)
LRRDLDA ENEAKKQVEKAL E
p*2.
(SEQ ID NO: 14)
LRRDLDA EDEAKKQVEKAL E
p*3.
(SEQ ID NO: 15)
LRRDLDA EREAKNQVEKAL E
p*4.
(SEQ ID NO: 16)
LRRDLDA EREAKKQVERAL E
p*5.
(SEQ ID NO: 17)
LRRDLDA EREAKKQVEMAL E
p*6.
(SEQ ID NO: 18)
LRRDLDA VNEAKKQVEKAL E
p*7.
(SEQ ID NO: 19)
LRRDLDA VDEAKKQVEKAL E
p*8.
(SEQ ID NO: 20)
LRRDLDA VREAKNQVEKAL E
p*9.
(SEQ ID NO: 21)
LRRDLDA VREAKKQVERAL E
p*10.
(SEQ ID NO: 22)
LRRDLDA VREAKKQVEMAL E
p*11.
(SEQ ID NO: 23)
LRRDLDA SNEAKNQVEKAL E
p*12.
(SEQ ID NO: 24)
LRRDLDA SNEAKKQVERAL E
p*13.
(SEQ ID NO: 25)
LRRDLDA SNEAKKQVEMAL E
p*14.
(SEQ ID NO: 26)
LRRDLDA SDEAKNQVEKAL E
p*15.
(SEQ ID NO: 27)
LRRDLDA SDEAKKQVERAL E
p*16.
(SEQ ID NO: 28)
LRRDLDA SDEAKKQVEMAL E
p*17
(SEQ ID NO: 6)
LRRDLDA SREAKNQVERAL E
p*18.
(SEQ ID NO: 29)
LRRDLDA SREAKNQVEMAL E

As used herein, a “J14 peptide” may comprise the amino acid sequence KQAEDKVKASREAKKQVEKALEQLEDKVK (SEQ ID NO:3) or a fragment or variant thereof, a peptide with minimal B and T cell epitopes within p145 that was identified as a GAS M protein C-region peptide devoid of potentially deleterious T cell autoepitopes, but which contained an opsonic B cell epitope. J14 is a chimeric peptide that contains 14 amino acids from M protein C-region (shown in bold) and is flanked by yeast-derived GCN4 sequences which was necessary to maintain the correct helical folding and conformational structure of the peptide.

As used herein a “J8 peptide” is a peptide which comprises an amino acid sequence at least partly derived from, or corresponding to, a GAS M protein C-region peptide. J8 peptide suitably comprises a conformational B-cell epitope and lacks potentially deleterious T-cell autoepitopes. A preferred J8 peptide amino acid sequence is QAEDKVKQSREAKKQVEKALKQLEDKVQ (SEQ ID NO:4) or a fragment or variant thereof, wherein the bolded residues correspond to residues 344 to 355 of the GAS M protein. In this embodiment, J8 is a chimeric peptide that further comprises flanking GCN4 DNA-binding protein sequences which assist maintaining the correct helical folding and conformational structure of the J8 peptide.

In other embodiments, the antibody or antibody fragment binds and/or is raised against a GAS superantigen.

As used herein a “superantigen” is a low molecular weight exo-protein that is secreted by all, or a substantial portion of, pathogenic GAS strains. There are 11 serologically distinct superantigens in GAS designated Spe-A, Spe-C, Spe-G, Spe-H, Spe-I, Spe-J, Spe-K, Spe-L, Spe-M, SSA, and SMEZ. Strepotococcal superantigens demonstrate high affinity binding to the human MHC II R chain and relatively low affinity binding to TCR β chains. Streptococcal superantigen protein structures show a conserved two-domain architecture and the presence of a long, solvent-accessible α-helix that spans the center of the molecule. The N-terminal domain is a mixed β-barrel with an oligonucleotide/oligosaccharide binding (OB) fold. The larger C-terminal domain is a β-grasp fold and consists of a twisted β-sheet that is capped by the central α4-helix that packs against a four-strand antiparallel twisted sheet. Streptococcal superantigens are extremely stable proteins that resist denaturing by heat and acid and this is achieved by close packing of the N- and C-terminal domains. The structure is further stabilized by a section of the N-terminus that extends over the top of the C-terminal domain. Notably, the most conserved section of all streptococcal superantigens is the region that builds the interface between the α4-helix and the inner side of the N-terminal OB-fold domain. Of the 11 superantigens that can be present in GAS, most cases of STTS are caused by one or other of streptococcal pyrogenic exotoxin (Spe) A or SpeC.

As used herein, a protein “variant” shares a definable amino acid sequence relationship with a reference amino acid sequence. The reference amino acid sequence may be an amino acid sequence of an M protein, superantigen or a fragment of these, as hereinbefore described. The “variant” protein may have one or a plurality of amino acids of the reference amino acid sequence deleted or substituted by different amino acids. It is well understood in the art that some amino acids may be substituted or deleted without changing the activity of the immunogenic fragment and/or protein (conservative substitutions). Preferably, protein variants share at least 70% or 75%, preferably at least 80% or 85% or more preferably at least 90%, 91%, 92%, 93%, 94%, 95%, 96%, 97%, 98% or 99% sequence identity with a reference amino acid sequence.

Non-limiting examples of p17 and/or p145 peptide variants are described in United States Patent Publication US2009/0162369, which is incorporated by reference herein.

Non-limiting examples of J8 peptide variants include:

(SEQ ID NO: 7)
SREAKKQSREAKKQVEKALKQVEKALC
(SEQ ID NO: 8)
SREAKKQSREAKKQVEKALKQSREAKC
(SEQ ID NO: 9)
SREAKKQVEKALKQSREAKKQVEKALC
(SEQ ID NO: 10)
SREAKKQVEKALDASREAKKQVEKALC

Other variants may be based on heptads such as described in Cooper et al., 1997, which is incorporated by reference herein.

By way of example, if an epitope is known to reside within an α-helix protein structural conformation, then a model peptide can be synthesised to fold to this conformation. We designed a model α-helical coiled coil peptide based on the structure of the GCN4 leucine zipper (O'Shea et al., 1991). The first heptad contains the sequence MKQLEDK (SEQ ID NO:11), which includes several of the features found in a stable coiled coil heptad repeat motif (a-b-c-d-e-f-g)n (Cohen & Parry, 1990). These include large apolar residues in the a and d positions, an acid/base pair (Glu/Lys) at positions e and g (usually favouring interchain ionic interactions), and polar groups in positions b, c, f (consistent with the prediction of Lupas etal. (1991)). The GCN4 peptide also contains a consensus valine in the a position. It has also been noted that when positions a and d are occupied by V and L a coiled coil dimer is favoured (Harbury et al., 1994). A model heptad repeat was derived from these consensus features of the GCN4 leucine zipper peptide: (VKQLEDK; SEQ ID NO:12) with the potential to form a α-helical coiled coil. This peptide became the framework peptide. Overlapping fragments of a conformational epitope under study were embedded within the model coiled coil peptide to give a chimeric peptide. Amino acid substitutions, designed to ensure correct helical coiled coil conformations (Cohen & Parry, 1990) were incorporated into the chimeric peptides whenever an identical residue was found in both the helical model peptide and the epitope sequence. The following substitutions were typically used: position a, V to I; b, K to R; c, Q to N; d, L to A; e, E to Q; f: D to E; g, K to R. All of these replacement residues are commonly found at their respective position in coiled coil proteins (Lupas et al., 1991).

Terms used generally herein to describe sequence relationships between respective proteins and nucleic acids include “comparison window”, “sequence identity”, “percentage of sequence identity” and “substantial identity”. Because respective nucleic acids/proteins may each comprise (1) only one or more portions of a complete nucleic acid/protein sequence that are shared by the nucleic acids/proteins, and (2) one or more portions which are divergent between the nucleic acids/proteins, sequence comparisons are typically performed by comparing sequences over a “comparison window” to identify and compare local regions of sequence similarity. A “comparison window” refers to a conceptual segment of typically 6, 9 or 12 contiguous residues that is compared to a reference sequence. The comparison window may comprise additions or deletions (i.e., gaps) of about 20% or less as compared to the reference sequence for optimal alignment of the respective sequences. Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, Wis., USA, incorporated herein by reference) or by inspection and the best alignment (i.e. resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA).

As used herein, “derivatives” are molecules such as proteins, fragments or variants thereof that have been altered, for example by conjugation or complexing with other chemical moieties, by post-translational modification (e.g. phosphorylation, acetylation and the like), modification of glycosylation (e.g. adding, removing or altering glycosylation), lipidation and/or inclusion of additional amino acid sequences as would be understood in the art. In one particular embodiment, an additional amino acid sequence may comprise one or a plurality of lysine residues at an N and/or C-terminus thereof. The plurality of lysine residues (e.g polylysine) may be a linear sequence of lysine residues or may be branched chain sequences of lysine residues. These additional lysine residues may facilitate increased peptide solubility. Another particular derivative is by conjugation of the peptide to diphtheria toxin (DT). This may be facilitated by addition of a C-terminal cysteine residue.

Additional amino acid sequences may include fusion partner amino acid sequences which create a fusion protein. By way of example, fusion partner amino acid sequences may assist in detection and/or purification of the isolated fusion protein. Non-limiting examples include metal-binding (e.g. polyhistidine) fusion partners, maltose binding protein (MBP), Protein A, glutathione S-transferase (GST), fluorescent protein sequences (e.g. GFP), epitope tags such as myc, FLAG and haemagglutinin tags.

Other additional amino acid sequences may be of carrier proteins such as diphtheria toxoid (DT) or a fragment thereof, or a CRM protein fragment such as described in International Publication WO2017/070735.

Other derivatives contemplated by the invention include, but are not limited to, modification to side chains, incorporation of unnatural amino acids and/or their derivatives during peptide, or protein synthesis and the use of crosslinkers and other methods which impose conformational constraints on the immunogenic proteins, fragments and variants of the invention.

In this regard, the skilled person is referred to Chapter 15 of CURRENT PROTOCOLS IN PROTEIN SCIENCE, Eds. Coligan et al. (John Wiley & Sons NY 1995-2008) for more extensive methodology relating to chemical modification of proteins.

The isolated M proteins, superantigen proteins, fragments and/or derivatives may be produced by any means known in the art, including but not limited to, chemical synthesis, recombinant DNA technology and proteolytic cleavage to produce peptide fragments.

Chemical synthesis is inclusive of solid phase and solution phase synthesis. Such methods are well known in the art, although reference is made to examples of chemical synthesis techniques as provided in Chapter 9 of SYNTHETIC VACCINES Ed. Nicholson (Blackwell Scientific Publications) and Chapter 15 of CURRENT PROTOCOLS IN PROTEIN SCIENCE Eds. Coligan et al., (John Wiley & Sons, Inc. NY USA 1995-2008). In this regard, reference is also made to International Publication WO 99/02550 and International Publication WO 97/45444.

Recombinant proteins may be conveniently prepared by a person skilled in the art using standard protocols as for example described in Sambrook et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. NY USA 1995-2008), in particular Chapters 10 and 16; and CURRENT PROTOCOLS IN PROTEIN SCIENCE Eds. Coligan et al., (John Wiley & Sons, Inc. NY USA 1995-2008), in particular Chapters 1, 5 and 6. Typically, recombinant protein preparation includes expression of a nucleic acid encoding the protein in a suitable host cell.

Certain aspects and embodiments of the invention relate to recombinant antibodies and antibody fragments which bind or are raised against M proteins, superantigen proteins, fragments and/or derivatives for administration to mammals for passive immunization against a Group A strep-associated disease of condition such as STSS. In a particular embodiment, the recombinant antibodies and antibody fragments are “humanized”, as hereinbefore described. Accordingly, some aspects of the invention provide of one or more isolated nucleic acids encoding recombinant antibodies and antibody fragments which bind or are raised against M proteins, superantigen proteins, fragments and/or derivatives.

The term “nucleic acid” as used herein designates single- or double-stranded DNA and RNA. DNA includes genomic DNA and cDNA. RNA includes mRNA, RNA, RNAi, siRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as modified purines (for example inosine, methylinosine and methyladenosine) and modified pyrimidines (for example thiouridine and methylcytosine).

In a preferred form, the one or more isolated nucleic acids encoding an M protein fragment, variant or derivative thereof and an agent that facilitates restoring or enhancing neutrophil activity are in the form of a genetic construct.

Suitably, the genetic construct is in the form of, or comprises genetic components of, a plasmid, bacteriophage, a cosmid, a yeast or bacterial artificial chromosome as are well understood in the art. Genetic constructs may also be suitable for maintenance and propagation of the isolated nucleic acid in bacteria or other host cells, for manipulation by recombinant DNA technology.

For the purposes of protein expression, the genetic construct is an expression construct. Suitably, the expression construct comprises the one or more nucleic acids operably linked to one or more additional sequences, such as heterologous sequences, in an expression vector. An “expression vector” may be either a self-replicating extra-chromosomal vector such as a plasmid, or a vector that integrates into a host genome.

By “operably linked” is meant that said additional nucleotide sequence(s) is/are positioned relative to the nucleic acid of the invention preferably to initiate, regulate or otherwise control transcription.

Regulatory nucleotide sequences will generally be appropriate for the host cell or tissue where expression is required. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the invention. The expression construct may also include an additional nucleotide sequence encoding a fusion partner (typically provided by the expression vector) so that the recombinant protein of the invention is expressed as a fusion protein, as hereinbefore described.

In a preferred form, the genetic construct is suitable for DNA vaccination of a mammal such as a human, by encoding the M protein and/or the superantigen described herein. In this regard, it will be appreciated that the M protein and the superantigen protein may be encoded on the same or different genetic constructs for vaccination purposes.

Suitably, the genetic construct is in the form of, or comprises genetic components of, a plasmid, bacteriophage, a cosmid, a yeast or bacterial artificial chromosome as are well understood in the art. Genetic constructs may also be suitable for maintenance and propagation of the isolated nucleic acid in bacteria or other host cells, for manipulation by recombinant DNA technology.

Suitably, DNA vaccination is by way of one or more plasmid DNA expression constructs. Plasmids typically comprise a viral promoter (such as SV40, RSV or CMV promoters). Intron A may be included to improve mRNA stability and thereby increase protein expression. Plasmids may further include a multiple cloning site, a strong polyadenylation/transcription termination signal, such as bovine growth hormone or rabbit beta-globulin polyadenylation sequences. The plasmid may further comprise Mason-Pfizer monkey virus cis-acting transcriptional elements (MPV-CTE) with or without HIV rev increased envelope expression. Additional modifications that may improve expression include the insertion of enhancer sequences, synthetic introns, adenovirus tripartite leader (TPL) sequences and/or modifications to polyadenylation and/or transcription termination sequences. A non-limiting example of a DNA vaccine plasmid is pVAC which is commercially available from Invivogen.

A useful reference describing DNA vaccinology is DNA Vaccines, Methods and Protocols, Second Edition (Volume 127 of Methods in Molecular Medicine series, Humana Press, 2006).

As hereinbefore described, the invention provides compositions, vaccines and/or methods of preventing or treating a Group A Strep-associated disease, disorder or condition in a mammal such as streptococcal toxic shock syndrome (STSS).

In the context of the present invention, by “group A-strep-associated disease, disorder or condition” is meant any clinical pathology resulting from infection by group A strep and includes cellulitis, erysipelas, impetigo, scarlet fever, throat infections such as acute pharyngitis (“strep throat”), bacteremia, streptococcal toxic shock syndrome (STSS), necrotizing fasciitis, acute rheumatic fever and acute glomerulonephritis, although without limitation thereto.

STSS is caused primarily by superantigen toxins that bind non-specifically to human MHC II molecules (outside the peptide binding groove) and T-cell receptor variable chains, resulting in polyclonal T-cell activation often with >20% of CD4+ T-cells being activated. This results in a Th1 cytokine storm which is the proposed causal link responsible for hypotension and multi-organ failure, (which includes the liver, kidney, coagulation system and respiratory system).

Suitably, the compositions and/or methods “passively immunize” the mammal against Group A Strep, or more particularly against STSS. Accordingly, administration of a combination of antibodies or antibody fragments that bind a group A Streptococcus M protein fragment or variant thereof and antibodies or antibody fragments that bind a group A Streptococcus superantigen fragment or variant thereof may confer, provide or facilitate at least partial passive immunity against subsequent infection by Group A Strep, or may confer, provide or facilitate at least partial passive immunity to an existing Group A Strep infection. It will also be appreciated that “passive immunity” does not exclude the elicitation of at least some elements of a host mammalian immune response such as induction of elements of the complement cascade, induction of elements of the innate immune system such as macrophages and other phagocytic cells and/or induction of cytokines, growth factors, chemokines and/or other pro-inflammatory molecules.

Suitably, passive immunization treats or prevents a Group A Strep-associated disease, disorder or condition in a mammal such as iGAS disease, inclusive of streptococcal toxic shock syndrome (STSS).

As used herein, “treating”, “treats” or “treatment” refers to a therapeutic intervention that at least partly ameliorates, eliminates or reduces a symptom or pathological sign of a Group A strep-associated disease, disorder or condition such as STSS, after it has begun to develop. Treatment need not be absolute to be beneficial to the mammal. The beneficial effect can be determined using any methods or standards known to the ordinarily skilled artisan.

As used herein, “preventing”, “prevents” or “prevention” refers to a course of action initiated prior to infection by, or exposure to, Group A strep and/or before the onset of a symptom or pathological sign of a Group A strep-associated disease, disorder or condition such as STSS, so as to prevent infection and/or reduce the symptom or pathological sign. It is to be understood that such preventing need not be absolute to be beneficial to a subject. A “prophylactic” treatment is a treatment administered to a subject who does not exhibit signs of a group A strep-associated disease, disorder or condition, or exhibits only early signs for the purpose of decreasing the risk of developing a symptom or pathological sign of a Group A strep-associated disease, disorder or condition.

In certain aspects and embodiments, the antibodies or antibody fragments that bind a group A Streptococcus M protein fragment or variant thereof and antibodies or antibody fragments that bind a group A Streptococcus superantigen fragment or variant, may be administered to a mammal separately, or in combination.

By “separately” is meant administered as discrete units respectively comprising the antibodies or antibody fragments that bind a group A Streptococcus M protein fragment or variant thereof and antibodies or antibody fragments that bind the group A Streptococcus superantigen fragment or variant at the same time, or which are temporally spaced apart in a manner which retains the combinatorial or synergistic efficacy of the respective antibodies or antibody fragments.

In some embodiments, the antibodies or antibody fragments that bind a group A Streptococcus M protein fragment or variant thereof and antibodies or antibody fragments that bind a group A Streptococcus superantigen fragment or variant, may be administered in the form of a composition.

In a preferred form, the composition comprises an acceptable carrier, diluent or excipient.

By “acceptable carrier, diluent or excipient” is meant a solid or liquid filler, diluent or encapsulating substance that may be safely used in systemic administration. Depending upon the particular route of administration, a variety of carriers, diluent and excipients well known in the art may be used. These may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates, water and pyrogen-free water.

A useful reference describing acceptable carriers, diluents and excipients is Remington's Pharmaceutical Sciences (Mack Publishing Co. N.J. USA, 1991) which is incorporated herein by reference.

Suitably, the M protein and/or superantigen protein described herein, inclusive of fragments, variants and derivatives thereof, are immunogenic. In the context of the present invention, the term “immunogenic” as used herein indicates the ability or potential to generate or elicit an immune response, such as to Group A strep or molecular components thereof, such as M protein or a superantigen, upon administration of the immunogenic protein or peptide to a mammal.

By “elicit an immune response” is meant generate or stimulate the production or activity of one or more elements of the immune system inclusive of the cellular immune system, antibodies and/or the native immune system. Suitably, the one or more elements of the immune system include B lymphocytes, antibodies and neutrophils.

Preferably, for the purposes of eliciting an immune response, certain immunological agents may be used in combination with the M protein, fragment, variant or derivative thereof, such as a J8 peptide, and/or the superantigen protein, fragment, variant or derivative, such as SpeA and SpeC, or with one or more genetic constructs encoding these.

The term “immunological agent” includes within its scope carriers, delivery agents, immunostimulants and/or adjuvants as are well known in the art. As will be understood in the art, immunostimulants and adjuvants refer to or include one or more substances that enhance the immunogenicity and/or efficacy of a composition. Non-limiting examples of suitable immunostimulants and adjuvants include squalane and squalene (or other oils of plant or animal origin); block copolymers; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); Bordetella pertussis antigens; tetanus toxoid; diphtheria toxoid; surface active substances such as hexadecylamine, octadecylamine, octadecyl amino acid esters, lysolecithin, dimethyldioctadecylammonium bromide, N,N-dicoctadecyl-N′, N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminium phosphate, aluminium hydroxide or alum; interleukins such as interleukin 2 and interleukin 12; monokines such as interleukin 1; tumour necrosis factor; interferons such as gamma interferon; immunostimulatory DNA such as CpG DNA, combinations such as saponin-aluminium hydroxide or Quil-A aluminium hydroxide; liposomes; ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran alone or with aluminium phosphate; carboxypolymethylene such as Carbopol′ EMA; acrylic copolymer emulsions such as Neocryl A640 (e.g. U.S. Pat. No. 5,047,238); water in oil emulsifiers such as Montanide ISA 720; poliovirus, vaccinia or animal poxvirus proteins; or mixtures thereof.

Immunological agents may include carriers such as thyroglobulin; albumins such as human serum albumin; toxins, toxoids or any mutant cross-reactive material (CRM) of the toxin from tetanus, diphtheria, pertussis, Pseudomonas, E. coli, Staphylococcus, and Streptococcus; polyamino acids such as poly(lysine:glutamic acid); influenza; Rotavirus VP6, Parvovirus VP1 and VP2; hepatitis B virus core protein; hepatitis B virus recombinant vaccine and the like. Alternatively, a fragment or epitope of a carrier protein or other immunogenic protein may be used. For example, a T cell epitope of a bacterial toxin, toxoid or CRM may be used. In this regard, reference may be made to U.S. Pat. No. 5,785,973 which is incorporated herein by reference.

Any suitable procedure is contemplated for producing vaccine compositions. Exemplary procedures include, for example, those described in New Generation Vaccines (1997, Levine et al., Marcel Dekker, Inc. New York, Basel, Hong Kong), which is incorporated herein by reference.

Any safe route of administration may be employed, including oral, rectal, parenteral, sublingual, buccal, intravenous, intra-articular, intra-muscular, intra-dermal, subcutaneous, inhalational, intraocular, intraperitoneal, intracerebroventricular, topical, mucosal and transdermal administration, although without limitation thereto.

Dosage forms include tablets, dispersions, suspensions, injections, solutions, syrups, troches, capsules, nasal sprays, suppositories, aerosols, transdermal patches and the like. These dosage forms may also include injecting or implanting controlled releasing devices designed specifically for this purpose or other forms of implants modified to act additionally in this fashion. Controlled release may be effected by coating with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions may be presented as discrete units such as capsules, sachets, functional foods/feeds or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

As generally used herein, the terms “patient”, “individual” and “subject” are used in the context of any mammalian recipient of a treatment or composition disclosed herein. Accordingly, the methods and compositions disclosed herein may have medical and/or veterinary applications. In a preferred form, the mammal is a human.

So that the invention may be fully understood and put into practical effect, reference is made to the following non-limiting Examples.

### EXAMPLES

### Introduction

When considering an antibody-based passive immunotherapy, it was appreciated that antibodies to the surface M protein (and to superantigens) are significantly lower in individuals who develop invasive disease [9] and that the low levels of antibodies in the general population may have contributed to the epidemic of invasive disease that started in the 1980s [10, 11]. However, it is not possible to determine whether the antibodies are low in individuals prior to the invasive infection or whether they become low after the infection commences as a result of antibody catabolism. We have reasoned that a direct way to address this issue is with an STSS model in which animals can be vaccinated and challenged or infected and treated. We have developed a GAS vaccine that is based on a highly conserved segment of the M protein (reviewed in [12]). The antigen is known as J8 and its sequence copies 12 amino acids of the C3-repeat of the M protein. Vaccination with J8 coupled to diphtheria toxoid (J8-DT) induces antibodies that opsonize GAS in vitro irrespective of the M-type and can protect mice from intraperitoneal and skin challenge [13-16]. However, as normal mice are not susceptible to superantigens (due to the very low affinity of mouse MHC II molecules to superantigens), it is not known whether this vaccine would prevent STSS. The work disclosed herein provides a suitable murine model to test the J8 GAS vaccine as a preventive measure pre-infection and passive immunotherapies using antibodies to J8 and to SpeA and SpeC as treatment options post infection.

### Materials and Methods

SN1→SN4 are clinical GAS isolates taken from the blood (×3) or wound swab (×1) of four adults who developed STSS in Brisbane at approximately the same time in 2015. Two of the four patients succumbed to their disease. The organisms were cultured in our laboratory with SN1 being used to develop the preliminary data set (below). Recombinant SpeC (rSpeC was purchased commercially from Toxin Tech (USA) and used in in vitro experiments and to generate anti-SpeC antibodies in mice. HLA-transgenic B6 mice (‘HLA-B6’) express HLA-DR3 and HLA-DQ2 [17].

The organisms were all emm 89 type. Genomic DNA was extracted from overnight stationary phase cultures using the GenElute bacterial gDNA extraction kit (Sigma). The gDNA was qualified using the Nanodrop1000 and then 2 μg of gDNA was used for amplification of all known superantigen genes. SDS-PAGE demonstrated that SN1-44 all contained the SpeC gene. They were also positive for SpeG and SmeZ but negative for Spes, A, L, M, H, I, J, K and ssa.

### Results

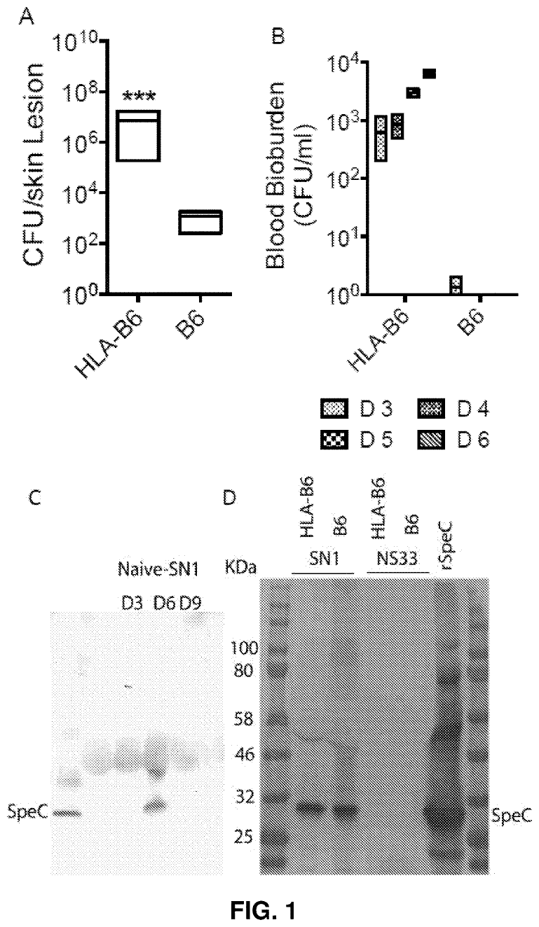

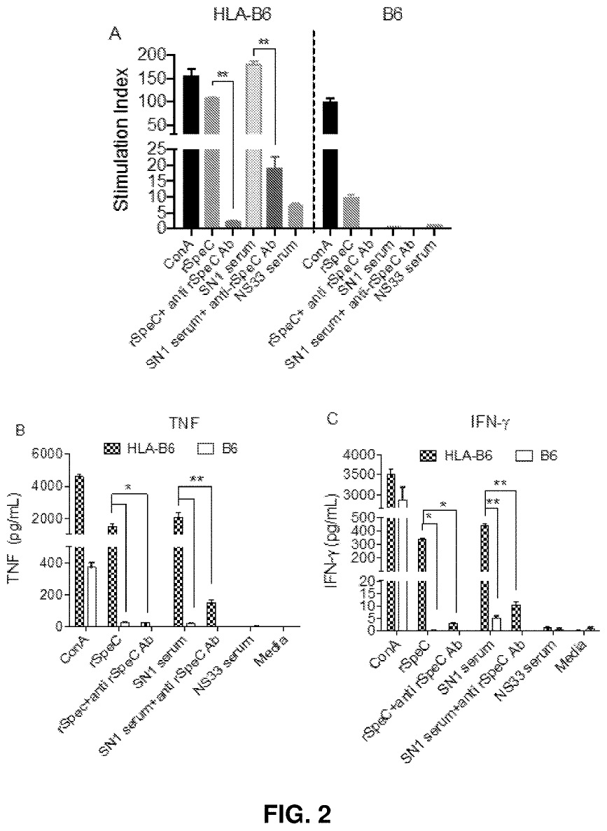

We found that HLA-B6 mice can develop iGAS disease following skin infection with non-mouse-adapted GAS strains (FIG. 1A-B). By contrast, GAS strains need to be adapted by serial passage before being able to cause iGAS disease in normal, non-humanized, mice. This may relate to the survival advantage that superantigens give GAS [18] and the necessity of human MHC II molecules for superantigens to be stimulatory. Thus, HLA-B6 mice should be ideal for modelling STTS. Nevertheless, following infection with SpeC-secreting GAS, BALB/c (non-HLA-transgenic) mice showed the presence of the SpeC toxin in their serum on day 6 post-infection (FIG. 1 C). This toxin-containing serum was sterile filtered and used as a reagent for in vitro and in vivo assays. HLA-B6 and wild-type control, C57/BL6 (B6) mice were infected with SN1 and with a group C Streptococcus (NS33) that does not express superantigens. Pooled serum samples from infected mice were collected at day 6 post-infection and run on a 4-15% gradient SDS-PAGE gel. Following protein transfer from the gel, the membrane was probed with primary antibody, Rabbit anti-SpeC IgG (Toxin-Tech, USA), followed by detection with Sheep anti-rabbit IgG-AP (Sigma-Aldrich) and developed using BCIP/NBT substrate (Sigma-Aldrich). rSpeC protein was also run as a positive control. SpeC was detected in the serum of SN1 infected mice whereas serum from NS33 infected mice did not show presence of toxin (FIG. 1 D).

SpeC-containing sera from infected BALB/c mice, or rSpeC, were added to splenocyte cultures of B6 or HLA-B6 mice. We observed significant proliferation of HLA-B6 splenic cells (but not spleen cells from B6 mice) in the presence of serum from infected mice or in the presence of rSpeC, but not in the presence of serum from mice infected with the group C Streptococcus (NS33) (FIG. 2 A). Proliferation was almost completely blocked by anti-rSpeC antibodies indicating that the other superantigens present in SN1 exerted minimal activity (FIG. 2 A). We observed similar responses when measuring the secretion of TNF and IFN-gamma (FIG. 2 B-C). Sera from infected BALB/c mice, or rSpeC, were also added to peripheral blood mononuclear cells (PBMC) of three healthy adult volunteers. We observed significant proliferation of the lymphocytes in all donors, in a dose-responsive manner (down to 5 μL per well) to serum from SN1-infected mice, but not to serum from NS33-infected mice. At 20 μL of SN1 serum per well, the proliferation of lymphocytes was similar to that induced by the mitogen, PHA. Proliferation was blocked by anti-rSpeC antibodies. These data demonstrate that SN1 expresses SpeC that is capable of non-specifically activating lymphocytes from HLA-humanized mice and from humans, consistent with the known pathogenesis of STSS. The data further suggest that HLA-B6 mice can be used to model STSS.

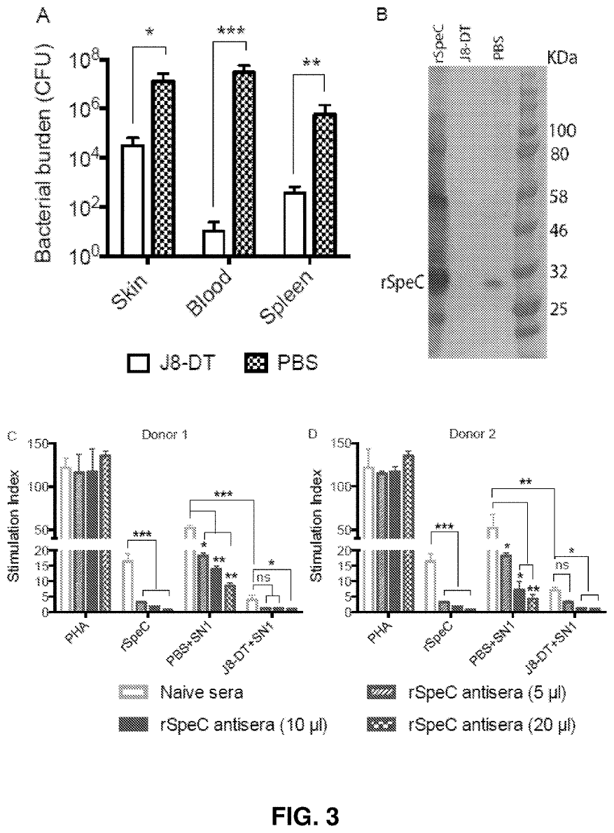

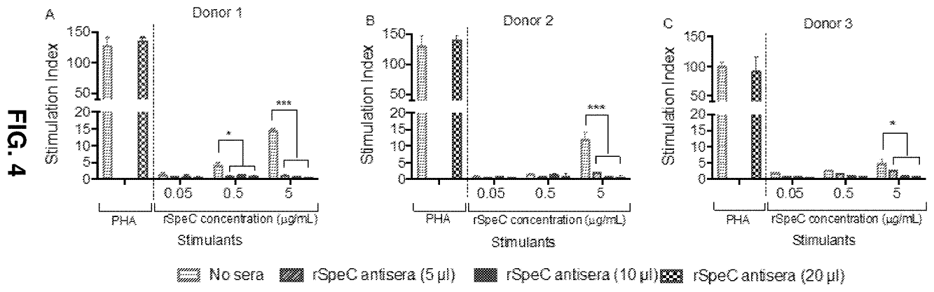

Prevention of mouse STSS via vaccination with J8. To determine whether vaccination with J8-DT will prevent STSS, we initially asked whether it would prevent skin and iGAS disease caused by SN1. Intramuscular vaccination (×3) of HLA-B6 mice with J8-DT/Alum reduced the bacterial burden in skin, blood and spleen by between 10,000 and 10,000,000-fold (FIG. 3 A). Western blot analysis of serum taken on day-6 post challenge demonstrated SpeC in the serum of control (PBS) mice, but not in the serum of J8-DT-vaccinated mice (FIG. 3 B).

We then tested whether serum from J8-DT-vaccinated SN1-infected mice would activate PBMCs taken from healthy volunteers. We observed that serum from non-vaccinated mice caused robust proliferation in 3 of 3 individuals (up to 50% of the level induced by PHA) but that serum from vaccinated mice resulted in significantly less proliferation. Representative data from 2 individuals are shown (FIG. 3 C-D). Similarly, antiserum to rSpeC significantly reduced the proliferative response caused by serum from SN1-infected mice. Furthermore, however, we observed that anti-rSpeC antisera (10-20 uL) added to the serum of mice from J8-DT-vaccinated HLA-B6 mice resulted in proliferation no greater than background levels (SI 1; P<0.05-0.01).

Development of a passive immunotherapy. A goal is to develop a combination passive immunotherapy consisting of antibodies to SpeA/C and antibodies to J8. Our preliminary data show that serum from BALB/c mice immunized with rSpeC can completely block the mitogenic effect of rSpeC on human PBMC when the toxin was added at 0.05 μg/ml, 0.5 μg/ml and 5 μg/ml (FIG. 4). The antiserum was effective down to a level of 5 μL per well.

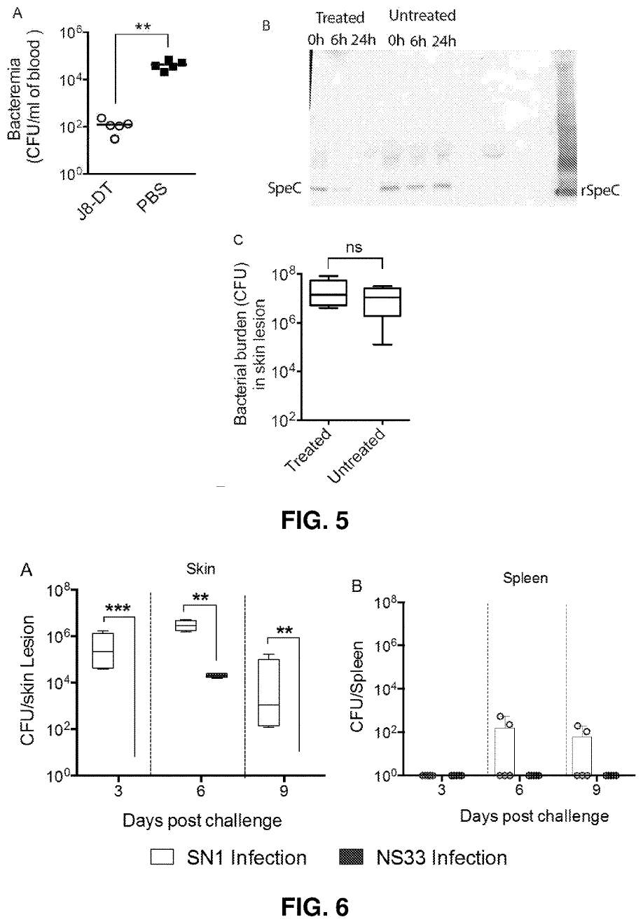

In the present Example, we have also shown the ability of J8-antisera to limit the development of STSS, but we have shown that J8-antisera (predominantly IgG1) from normal mice can rapidly reduce the bacterial bioburden in recipient animals (FIG. 5 A). However, our data show that a combination of anti-SpeC and anti-J8 antibodies are superior in that they neutralize both SpeC and the M protein and by including anti-J8 antibodies, they also remove the bacteria from the circulation. We also saw that anti-SpeC antisera administered to BALB/c mice 5 days after infection can neutralize SpeC within 6 h of administration (FIG. 5 B). However this treatment did not lead to a reduction in skin bacterial burden (FIG. 5 C).

### Further Proposed Studies

We have shown that iGAS disease can develop in HLA-B6 mice following infection with non-mouse-adapted GAS strains and that lymphocytes from HLA-B6 mice respond to SpeC from SN1 GAS in a manner consistent with the pathogenesis of STSS. To extend the research, we will firstly ask whether other strains of GAS that we have in our collection and which we know from genomic screens to be SpeC POS (Table 1), will also activate lymphocytes from HLA-B6 mice. We will determine, via western blot, the presence of SpeC in the serum of HLA-B6 mice infected with 4 additional SpeC POS GAS strains. Splenocytes from non-infected HLA-B6 mice (n=5/GAS isolate) will then be cultured with rSpeC or serum from mice infected with the different SpeC-POS strains. Lymphocyte proliferation and secreted TNF and IFN-gamma will be measured as above. Briefly, naïve splenocytes will be stimulated with pre-optimised concentration of serum from SpeC POS GAS-infected mice. Proliferation will be measured by [³H] thymidine uptake after 72 h. Cell-free culture supernatants will be tested for various cytokines using a CBA kit (BD Biosciences). Normal mouse serum (NMS) and serum from superantigen-NEG NS33 group C streptococcal infected mice will be used as negative controls. Experiments will be repeated at least twice, We will also collect GAS clinical samples and test these as they become available.

SpeC is one of two major superantigens from GAS, the other being SpeA. We will similarly test the ability of 5 different SpeA POS GAS strains (Table 1) and new samples (GAS isolates and serum from STSS patients) to activate spleen cells from HLA-B6 mice.

SpeA is known to bind to HLA DR4 and DQ8 [18]; however, is also binds DR3 and DQ2, indicating that the HLA-B6 mice which we currently possess will be suitable for STSS studies with SpeA-bearing GAS. We will use rSpeA as a positive control. It will be purchased from ToxinTech, USA.

We have previously developed a skin challenge model [14]. By topically inoculating streptococci to lightly abraded skin, this model closely replicates human pyoderma. Given that most cases of STTS commence from skin, this is the ideal challenge model. Bacterial burden can be quantified accurately by euthanizing mice and estimating the number of colonies in homogenised excised skin. The invasive bacterial burden is determined by plating blood and homogenized spleen samples. Using this model, we have shown that intramuscular vaccination of different strains of normal mice with J8-DT/Alum (×3) can protect against GAS pyoderma and iGAS disease in a serotype-independent manner.

HLA-B6 mice will be vaccinated (intramuscular×3) with J8-DT/Alum (or PBS/Alum as a control) on days 0, 21 and 42. Two weeks post vaccination; mice will be challenged via the skin with 5 different SpeA POS and 5 different SpeC POS GAS strains. Group sizes of 15 animals will be used. Mice will be observed for signs of clinical disease over the course of 9 days. The bacterial burdens in skin, blood and spleen will be estimated by euthanizing a designated number of mice (n=5 mice/group) at days 3, 6 and 9 post-challenge. Serum samples from blood collected at various time-points will be used to determine the presence of SpeA and SpeC, via western blot. Presence of elevated levels of liver enzymes (as an indicator of hepatic damage) will be investigated as previously described [19]. Sera from vaccinated and control mice will be sterile-filtered and tested for their ability to stimulate lymphocyte proliferation and cytokine secretion by spleen cells from HLA-B6 mice and human PBMC. [³H] Thymidine uptake assay and CBA kit will be utilised to measure proliferation and cytokine secretion respectively.

We will thus have three readouts for protection from STSS: (i) clinical and serological analysis of vaccinated infected mice; (ii) prevention of stimulation of splenocytes from HLA-B6 mice in vitro following incubation with filtered serum from vaccinated vs control mice; and (iii) prevention of stimulation of PBMC from normal human volunteers following incubation with filtered serum from vaccinated vs control mice.

IVIG has been shown to significantly improve survival for STSS and this has been attributed to the presence of antibodies to streptococcal superantigens. Additionally, naturally acquired antibodies to superantigens and the M protein have been suggested to be responsible for protection against STSS.

We will test a combination of anti-SpeA/C and anti-J8 antibodies for protection against streptococcal bioburden and SpeA/C-mediated lymphocyte stimulation following infection of HLA-B6 mice with SpeA POS GAS and SpeC POS GAS. Initially, the experiments will be performed without antibiotic co-therapy. Monoclonal antibodies against SpeA, SpeC and J8 will be produced. For monoclonal antibody production, superantigen proteins SpeA and SpeC will be commercially sourced from Toxin Technology Inc. FL USA. Our preliminary data show that anti-J8 antiserum (IgG1 isotype) can reduce bacterial bioburden in recipient mice by almost 1000-fold within 48 hours (FIG. 5 A) and that anti-SpeC antisera administered to BALB/c mice 6 days after infection can neutralize SpeC within 6 h of administration (FIG. 5 B). However anti-SpeC antiserum treatment did not lead to a reduction in skin bacterial burden, suggesting the need for opsonic activity of J8 antibodies (FIG. 5 C). IgG1 monoclonal antibodies will be tested alongside antisera to the recombinant superantigens and to J8. Activity will be defined as absence of the 26 kDa superantigen band on WB of sera from infected mice (for the superantigen MAbs and the J8 MAbs) and reduced bioburden after 24 hours of treatment (for the J8 MAbs). The optimal amount of antibody required for significant SpeA/C blocking or reduction in bioburden will be determined. The most active blockers will be carried forward for combination immunotherapy studies using the optimal determined dose of antibody.

HLA-B6 mice (10 per group) will be infected with SpeA POS or SpeC POS GAS. Mice will then receive, either anti-J8 antibody alone, anti-SpeA/C antibody alone, a combination of both or control isotype-matched Mab via the intravenous route. Mice will then be observed for clinical symptoms and blood taken daily to estimate bacterial burden and the presence/absence of SpeA/C in blood. Sera collected at various time-points post treatment will be used to test whether they activate lymphocytes from HLA-B6 mice and human volunteers (indicating the presence of superantigens). Sera will also be used to measure liver enzyme levels. STSS can cause liver dysfunction resulting in jaundice and high levels of aminotransferases due to hypo perfusion and circulating toxins. It is expected that significant improvement in all parameters will be observed within 24 hours. Therapies that have a positive clinical benefit will then be administered to another cohort of mice that will receive antibiotic therapy (penicillin) [20] to determine whether immunotherapy can hasten recovery.

A number of the above mentioned studies have been performed in Example 2, outlined below.

### SUMMARY

STSS and iGAS disease are increasing in prevalence annually and affect all sectors of society, although marginalised populations bear the brunt of the epidemic. The current best treatment option for STSS is IVIG and antibiotic therapy. While IVIG is expensive and of variable quality, it does provide good evidence that streptococcal-specific antibodies, in conjunction with antibiotics, are required for treatment. Our preliminary data provide strong evidence that antibodies to the M protein and to specific toxins will be the best line of treatment. J8-specific antibodies can neutralize GAS, and by targeting the conserved region of the M protein, have the distinct advantage of being protective against all strains. SpeC toxin-specific antibodies can neutralise the toxin and provide proof-of-principle that antibodies to the other major toxin, SpeA will do the same. These two toxins are responsible for most cases of STSS. A combination of an immune response targeting the organism (anti-J8) with one that neutralizes the major toxins (anti-SpeA, anti-SpeC) provides an innovative step that has not been tested or developed previously, but one in which we have significant abilities and experience to develop further and to eventually take to the clinic.

TABLE 1
List of GAS isolates expressing SpeA or SpeC toxin.

| Nos | Isolate | emm-type | Source | SpeA | SpeC |
|---|---|---|---|---|---|
| 1 | SN1 | 89 | blood | NEG | POS |
| 2 | NS1 | 100 | skin | NEG | POS |
| 3 | NS7 | 80 | blood | NEG | POS |
| 4 | NS12 | 61 | blood | NEG | POS |
| 5 | NS35 | 53 | axilla abscess swab | NEG | POS |
| 6 | NS24 | 24 | blood | POS | NEG |
| 7 | NS25 | 12 | blood | POS | NEG |
| 8 | 5448 | 1 | blood | POS | NEG |
| 9 | 88/373 | 49 | blood | POS | NEG |
| 10 | HKU425 | 1 | left anterior arm | POS | NEG |
| | | | deep fasciitis tissue | | |

### REFERENCES

1. Pahlman, L. I., et al., Streptococcal M protein: a multipotent and powerful inducer of inflammation. J Immunol, 2006. 177(2): p. 1221-8.
2. Faulkner, L., et al., The mechanism of superantigen-mediated toxic shock: not a simple Th1 cytokine storm. J Immunol, 2005. 175(10): p. 6870-7.
3. DaSilva, L., et al., Humanlike immune response of human leukocyte antigen-DR3 transgenic mice to staphylococcal enterotoxins: a novel modelfor superantigen vaccines. J Infect Dis, 2002. 185(12): p. 1754-60.
4. McCormick, J. K., et al., Development of streptococcal pyrogenic exotoxin C vaccine toxoids that are protective in the rabbit model of toxic shock syndrome. J Immunol, 2000. 165(4): p. 2306-12.
5. Roggiani, M., et al., Toxoids of streptococcal pyrogenic exotoxin A are protective in rabbit models of streptococcal toxic shock syndrome. Infect Immun, 2000. 68(9): p. 5011-7.
6. Kaul, R., et al., Intravenous immunoglobulin therapy for streptococcal toxic shock syndrome—a comparative observational study. The Canadian Streptococcal Study Group. Clin Infect Dis, 1999. 28(4): p. 800-7.
7. Linner, A., et al., Clinical efficacy of polyspecific intravenous immunoglobulin therapy in patients with streptococcal toxic shock syndrome: a comparative observational study. Clin Infect Dis, 2014. 59(6): p. 851-7.
8. Jolles, S., W. A. Sewell, and S. A. Misbah, Clinical uses of intravenous immunoglobulin. Clin Exp Immunol, 2005. 142(1): p. 1-11.
9. Basma, H., et al., Risk factors in the pathogenesis of invasive group A streptococcal infections: role of protective humoral immunity. Infect Immun, 1999. 67(4): p. 1871-7.
10. Holm, S. E., et al., Aspects of pathogenesis of serious group A streptococcal infections in Sweden, 1988-1989. J Infect Dis, 1992. 166(1): p. 31-7.
11. Stevens, D. L., Invasive group A Streptococcus infections. Clin Infect Dis, 1992. 14(1): p. 2-11.
12. Good, M. F., et al., Strategic development of the conserved region of the M protein and other candidates as vaccines to prevent infection with group A streptococci. Expert Rev Vaccines, 2015. 14(11): p. 1459-70.
13. Batzloff, M. R., et al., Protection against group A Streptococcus by immunization with J8-diphtheria toxoid: contribution of J8- and diphtheria toxoid-specific antibodies to protection. J Infect Dis, 2003. 187(10): p. 1598-608.
14. Pandey, M., et al., A synthetic M protein peptide synergizes with a CXC chemokine protease to induce vaccine-mediated protection against virulent streptococcal pyoderma and bacteremia. J Immunol, 2015. 194(12): p. 5915-25.
15. Pandey, M., et al., Combinatorial Synthetic Peptide Vaccine Strategy Protects against Hypervirulent CovR/S Mutant Streptococci. J Immunol, 2016. 196(8): p. 3364-74.
16. Pandey, M., et al., Physicochemical characterisation, immunogenicity and protective efficacy of a lead streptococcal vaccine: progress towards Phase I trial. Sci Rep, 2017. 7(1): p. 13786.
17. Chen, Z., et al., A 320-kilobase artificial chromosome encoding the human HLA DR3-DQ2 MHC haplotype confers HLA restriction in transgenic mice. J Immunol, 2002. 168(6): p. 3050-6.
18. Kasper, K. J., et al., Bacterial superantigens promote acute nasopharyngeal infection by Streptococcus pyogenes in a human MHC Class II-dependent manner. PLoS Pathog, 2014. 10(5): p. e1004155.
19. Ukpo, G. E., O. A. Ebuehi, and A. A. Kareem, Evaluation of Moxifloxacin-induced Biochemical Changes in Mice. Indian J Pharm Sci, 2012. 74(5): p. 454-7.
20. Gonczowski, L. and G. Turowski, The effect of penicillin on skin graft survival in mice. Arch Immunol Ther Exp (Warsz), 1984. 32(3): p. 351-6.

### Example 2

### Introduction:

Seemingly mild streptococcal infections can rapidly escalate to serious invasive infections with a high mortality rate. The overall incidence reported for invasive group A streptococcal disease (ISD) varies between 2-4 per 100,000 people in developed countries. However, most of these data were garnered from multiple surveys conducted between 1996 and 2007 [1, 2]. A study from the USA covering the 2005-2012 period showed a steady rate of 3.8 per 100,000 [3]. Periodic upsurges in incidence rates have previously been described in various countries, but the most recent reports show a worrying and sustained increase in incidence throughout Canada, particularly from 2013 (Public health Agency of Canada). In Alberta the rates have dramatically increased from 4.2 per 100,000 in 2003 to 10.2 per 100,000 in 2017 [4]. Very high rates are also reported amongst the young and the elderly, and particularly from developing countries. For example, the incidence rates amongst indigenous Fijians were reported to be approximately 60 per 100,000 in young children and 75 per 100,000 in the elderly in 2007 [2]. The true current global incidence rates are unknown, but available data point to rates being significantly higher than those reported.

In approximately 20% of cases, ISD is accompanied by a streptococcal toxic shock syndrome (STSS) with multi-organ failure and case fatality rates in excess of 40%, even in the best-equipped facilities [5]. It can occur after any streptococcal infection but most commonly occurs after infections of the skin and is usually associated with necrotising fasciitis, myositis or deep bruising. Pregnancy and the puerperium are periods of excessive risk, especially in developing countries [6].

Streptococcal ‘superantigens’ (SAgs) are thought to play a key role in the pathogenesis of STSS. These exotoxins are secreted by all pathogenic Streptococcus pyogenes and Staphylococcus aureus strains [7]. Nine of the 11 streptococcal SAg genes are located in bacteriophages. The phage-encoded Streptococcal pyrogenic exotoxin (Spe) A and SpeC are responsible for most cases of STSS. SAgs have profound immunological potency that is derived from their non-specific binding to human MHC (HLA) Class II molecules (outside the peptide binding groove) and conserved regions of the T-cell receptor chains, resulting in polyclonal T-cell activation often with >25% of CD4+ T-cells being activated. The resulting Th1 cytokine storm is the proposed causal link responsible for the hypotension and multi-organ failure that defines STSS. This has led to toxoids of SAgs being proposed as vaccine candidates [8, 9]. However, the pathogenesis of STSS is not fully understood. Other streptococcal virulence factors, including SLO [10], peptidoglycan, lipoteichoic acid [11, 12] and the M protein [13] have been shown to be potent inducers of inflammatory cytokines in vitro, and these or other factors may play important roles in STSS and be key to the development of successful vaccines and immunotherapies.

‘J8’ is a vaccine candidate based on the highly conserved C-3 repeat region of the M protein. It can protect mice from skin, mucosal and intraperitoneal streptococcal sepsis via antibody-mediated neutrophil opsonophagocytosis [14-16]. When conjugated to diphtheria toxoid (DT), it is immunogenic in non-human primates [17] and in humans [18] and is currently undergoing further clinical trials to study immunogenicity and efficacy.

In the present Example, HLA DR3 DQ2-transgenic mice were used to model STSS and asked whether vaccination with J8 could prevent STSS-like disease and whether passive immunotherapy with J8- and SpeC-specific antibodies could treat established STSS. The data demonstrate critical roles for both the SAg and the M protein in pathogenesis and show that antibodies to both, acting cooperatively, completely negate both the clinical signs of disease and the associated potent mitogenic activity of a Strep A organism isolated from a patient who succumbed to STSS.

Results

Establishing a Humanized Mouse Model for STSS

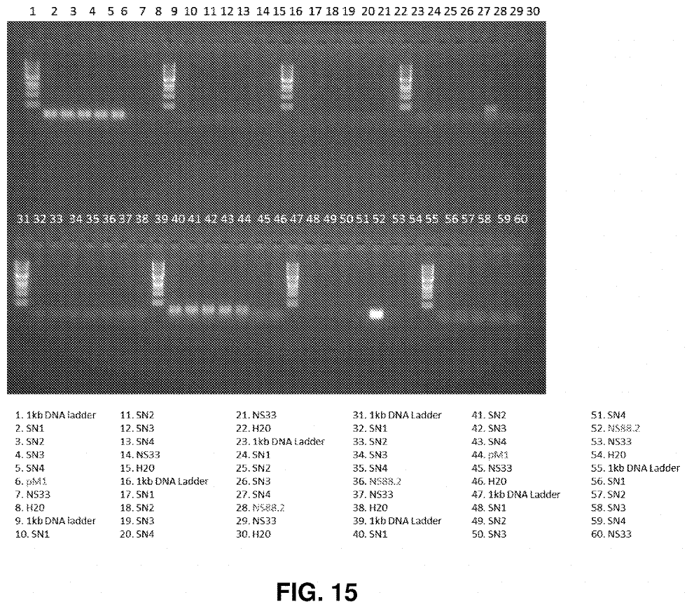

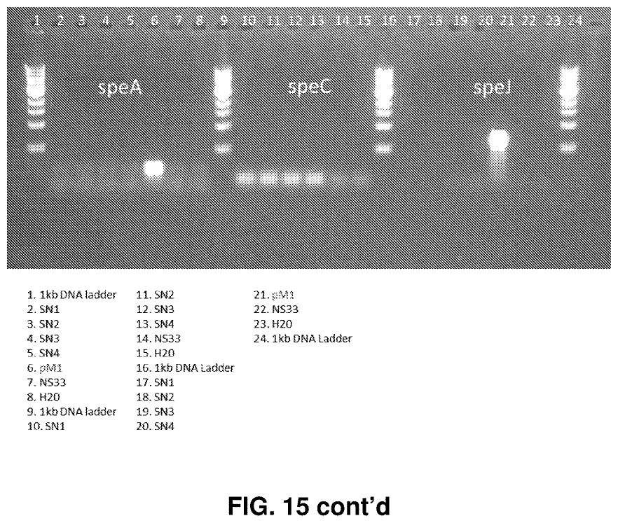

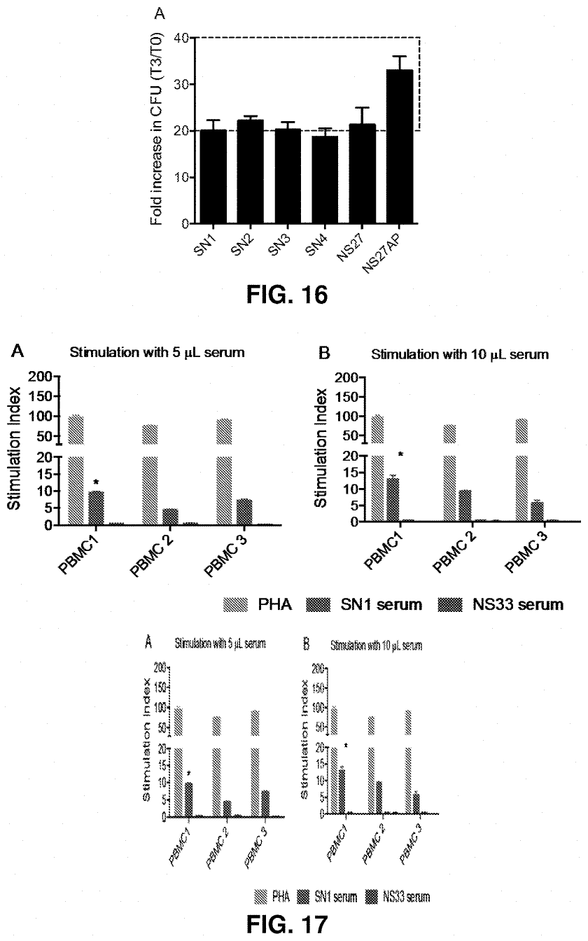

SN1 is an emm 89 strain of S. pyogenes (group A Streptococcus) isolated in 2015 from the blood of a patient in Brisbane who experienced STSS and succumbed to the disease. Genomic analysis revealed that from all known 11 streptococcal SAg genes examined, SN1 expressed the phage-encoded SpeC gene, and the chromosomally encoded SmeZ and SpeG genes (FIG. 15). The organism was negative for SpeA. Another group A Streptococcus (NS33) (isolated from a patient with foot ulcer-sourced from Royal Darwin Hospital) did not express any SAg genes.

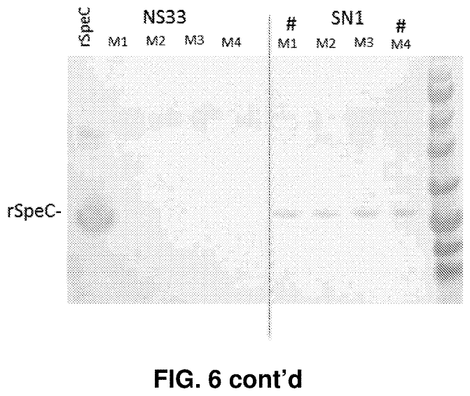

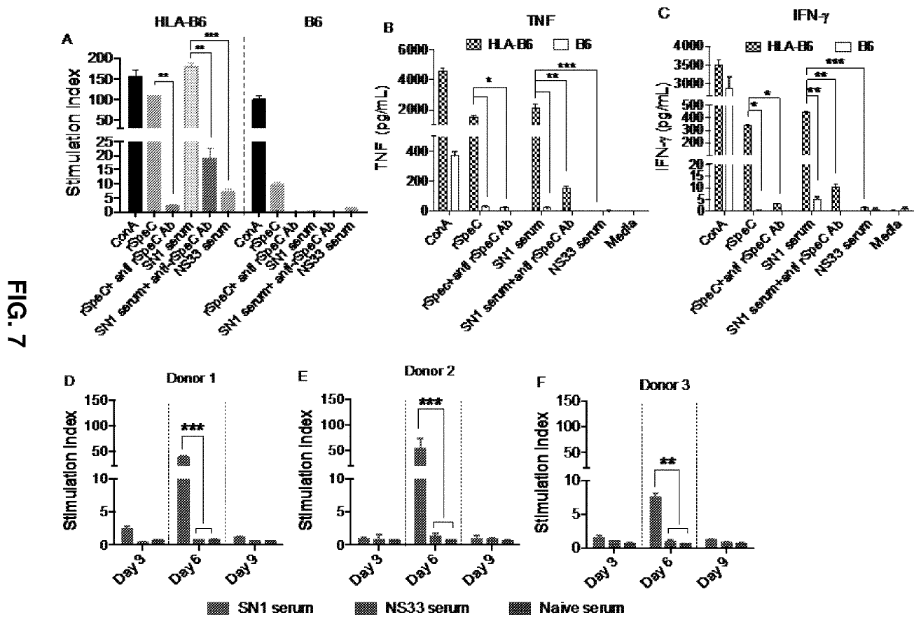

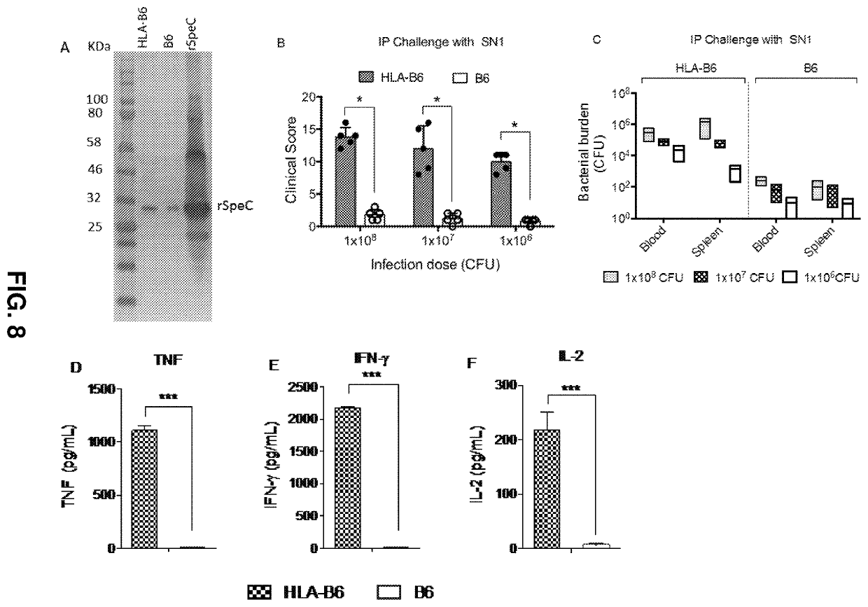

BALB/c mice were infected via skin scarification with SN1 or NS33. These mice developed a skin infection but did not develop a systemic infection and did not become ill; however, blood samples collected from SN1-infected mice were positive for the SpeC toxin as determined by western blot (WB) analysis. SpeC was not detected in blood of mice infected NS33 (FIG. 6 A-C).

Sera from BALB/c mice infected via the skin with SN1, or recombinant SpeC from E. coli (recSpeC) protein, were added to B6 or HLA-transgenic B6 splenocyte cultures. We observed significant proliferation of spleen cells from HLA-B6 mice (but not from B6 mice) to both the serum and recSpeC (FIG. 7 A). Proliferation was not completely blocked by anti-recSpeC antibodies indicating that the other molecules present in SN1 exerted some mitogenic activity (FIG. 7 A). The proliferative responses were mirrored by the production of TNF and IFN-γ—two key cytokines implicated in STSS pathogenesis (FIG. 7 B-C). Serum from NS33-infected mice did not induce any proliferation in splenocytes from either HLA-B6 or B6 mice.

The human relevance of these responses was confirmed when sera from the infected BALB/c mice, or recSpeC, were added to peripheral blood mononuclear cells (PBMCs) from three healthy adult volunteers. We observed significant proliferation of lymphocytes in all donors, in a dose responsive manner (down to 5 ul per well) to serum from SN1-infected mice, but not from NS33-infected mice (FIG. 17). We also observed that the day 6 sera from SN1 infected mice caused maximum proliferation of human lymphocytes (FIG. 7 D-F), which correlated with the presence of SpeC toxin in the serum at that time (FIG. 6 C). These data demonstrated that SN1 expresses SpeC that is capable of non-specifically activating lymphocytes from HLA-B6 mice and from humans, consistent with the known pathogenesis of STSS.

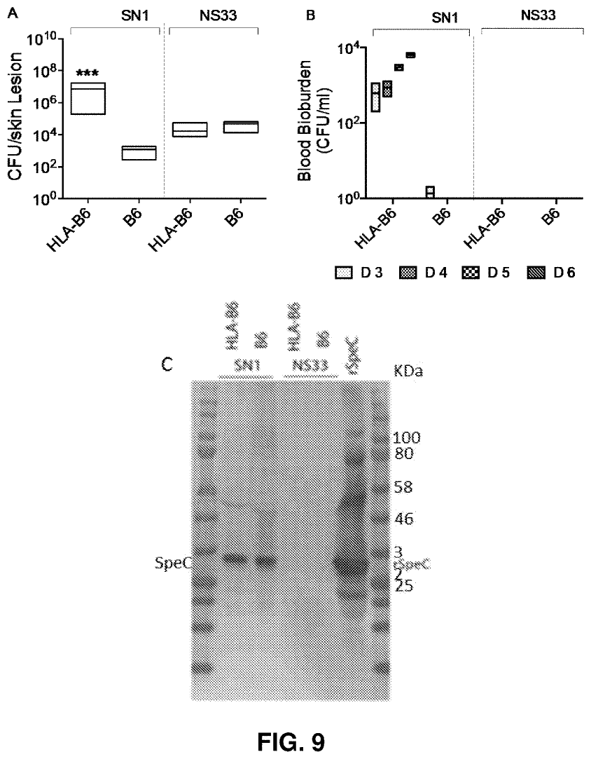

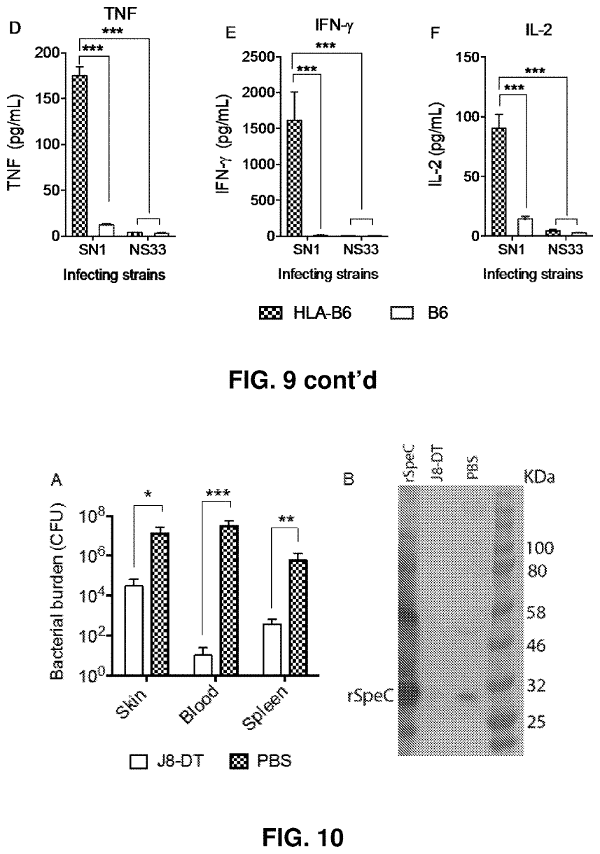

We next assessed the clinical virulence of SN1 in HLA-B6. Mice were infected intraperitoneally with varying doses of SN1 (10⁶, 10⁷, or 10⁸ CFU). SpeC was detected in the sera of mice infected with 1×10⁶ CFU, 24 hours post infection (FIG. 8 A). At this time, they demonstrated clinical symptoms (FIG. 8 B) and were euthanized (in accordance with an approved Ethics committee protocol). Bacterial burdens were assessed in blood and spleen (FIG. 8 C). We observed a dose-dependent infection outcome with clinical scores directly related to bacterial burden. (FIG. 8 B-C). High levels of TNF, IFN-γ and IL-2 were detected in the sera of infected mice (FIG. 9 D-F).

We asked if skin infection of HLA-B6 mice would also cause STSS-like pathology. On day 6-post infection with 1×10⁶ CFU, HLA-B6 mice demonstrated significantly higher bacterial burden in the skin lesion compared to B6 mice (FIG. 9 A). These mice also developed septicaemia although the bacterial burden was much lower than in mice infected via the intraperitoneal route (FIG. 9 B). Infection with NS33 in both HLA-B6 and B6 mice resulted in a modest local infection (103-104 CFU/skin lesion) with no septicaemia (FIG. 9 A-B). SpeC was detected in their blood (FIG. 9C), which also contained high levels of TNF, IFN-γ and IL-2. Neither skin infection of HLA-B6 mice with NS33 nor B6 mice with either SN1 or NS33, resulted in cytokine induction (FIG. 9 D-F).

Vaccine Prevention of STSS

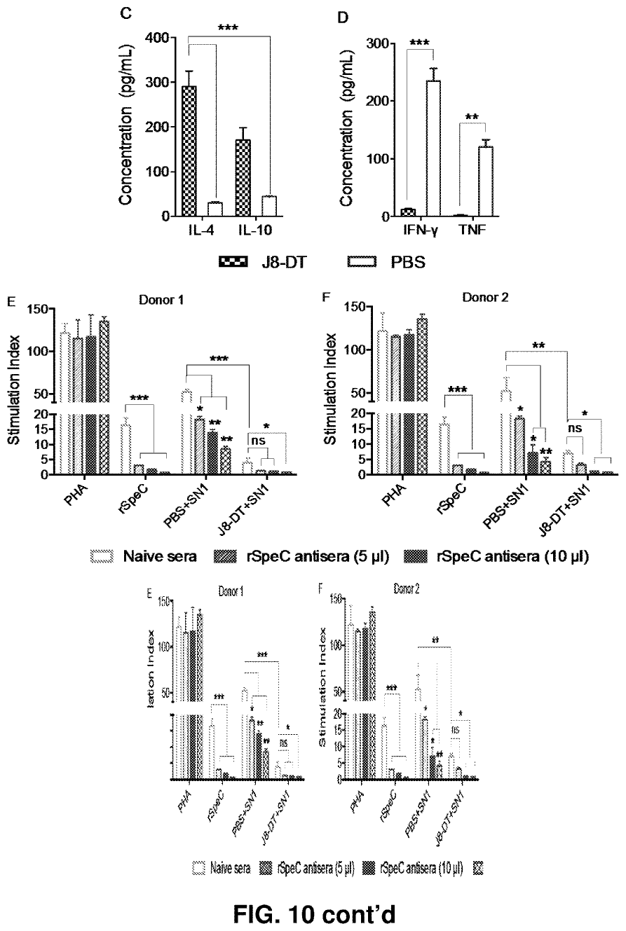

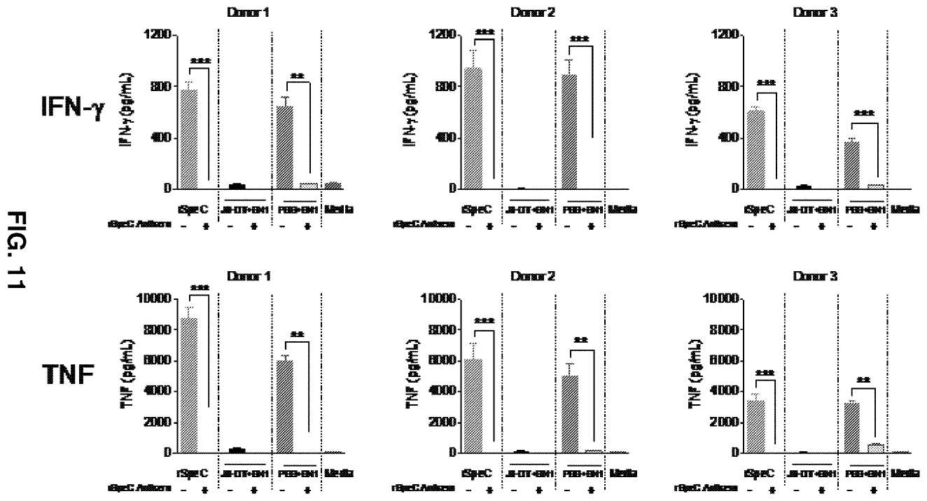

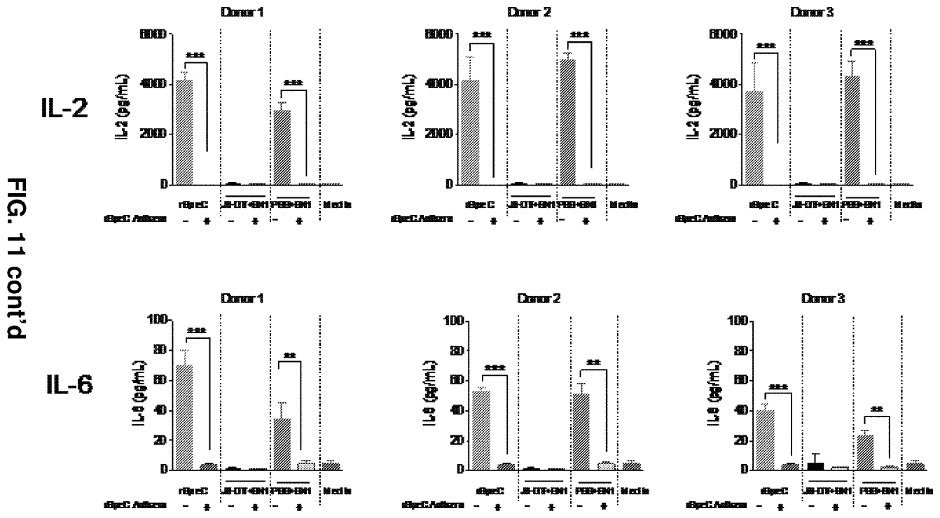

Having shown that HLA-B6 mice develop an STSS-like pathology following either superficial or systemic infection with SN1, we asked whether this could be prevented by vaccination with J8 coupled to diphtheria toxoid and administered with Alum (J8-DT/Alum). Vaccinated mice demonstrated a 1000-1,000,000-fold reduced bacterial burden in skin, blood and spleen following a skin challenge infection with SN1 (P values, <0.05, <0.001 and <0.01, respectively) (FIG. 10 A). SpeC was detected in the serum of control mice vaccinated with PBS but not in the serum of mice vaccinated with J8-DT (FIG. 10 B). The Th1 cytokines, IFN-γ and TNF, were also detected in the serum of control mice whereas the Th2 cytokines, IL-4 and IL-10, were found in the serum of protected mice (FIG. 10C,D).

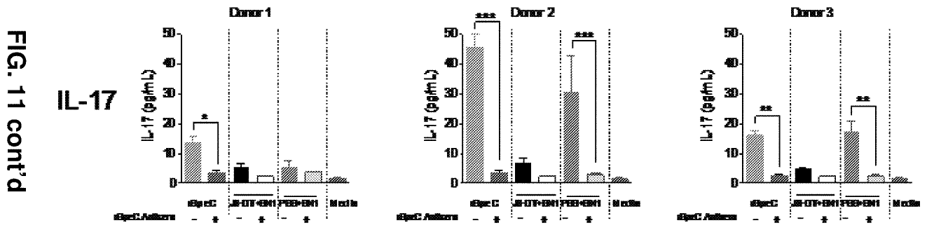

Filtered sera from J8-DT-vaccinated and infected, and control (PBS-vaccinated/infected) mice, or recSpeC, were added to cultures of human PBMCs from 3 healthy individuals. Serum from PBS-vaccinated/infected mice caused robust proliferation in PBMC from all individuals (up to 50% of the level induced by PHA). This was largely due to bacterial SpeC present in the serum as addition of recSpeC antiserum reduced the level of T cell activation by between 80-90% in a dose dependent manner (FIG. 10 E-G). Serum from J8-DT-vaccinated/infected mice caused significantly less cell proliferation compared to serum from PBS-vaccinated/infected mice (90-95% reduction) and this was further reduced to background levels by addition of recSpeC antiserum (Stimulation Index ˜1; p<0.05-0.01) (FIG. 10 E-G). This indicated that there was still residual SpeC present in the serum of J8-DT-vaccinated/infected mice, even though this was not apparent from inspection of the WB (FIG. 10B). Consistent with the proliferation data, the PBMC induction of inflammatory cytokines (IFN-γ, TNF, IL-2, IL-6, IL-17) by serum from J8-DT-vaccinated/infected mice was significantly reduced compared to the production of cytokines by serum from PBS-vaccinated/infected mice (FIG. 11). The responses induced by PBS-vaccinated/infected sera were comparable to the responses induced by recSpeC. These data thus demonstrate that streptococcal SpeC is responsible for >90% of all T cell activation and cytokine responses observed in vitro following SN1 infection and that prior J8-DT vaccination can prevent >90% of the in vitro responses that occur as a result of serum mitogenic factors. While we have previously shown that vaccination with J8-DT can significantly reduce bacterial burden following challenge, the data in FIGS. 10 and 11 do not exclude a separate role for anti-J8 antibodies which could be having a direct effect on the M protein of SN1 and block any mitogenic effect that it may have.

Immunotherapy for STSS

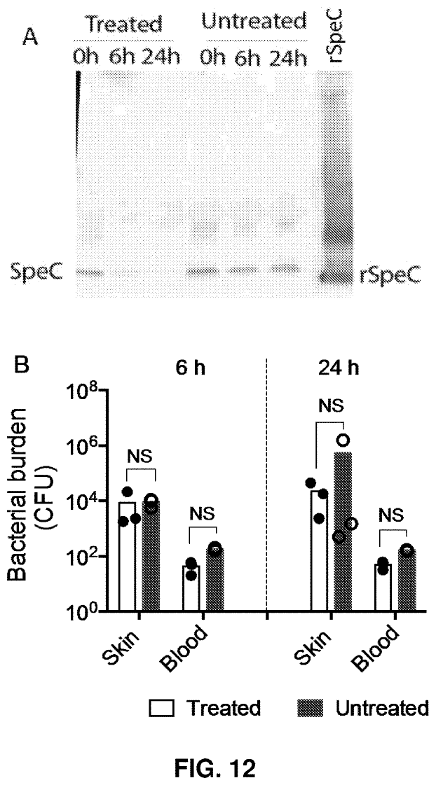

To assess the therapeutic efficacy of and recSpeC antisera, HLA-B6 mice were infected with SN1 via the skin and were treated with antisera (or naïve serum) on day 5 post-infection. SpeC was present in the serum of infected mice prior to treatment but was significantly reduced when measured at 6 hrs and absent at 24 hrs. It was present in control mice when measured at 6 and 24 h (FIG. 12A). Treatment with anti-SpeC antiserum did not diminish the bacterial burden in the skin or blood relative to those mice receiving naïve serum (FIG. 12 C).

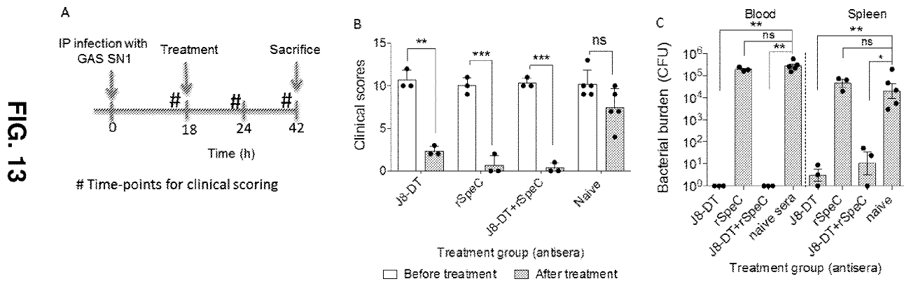

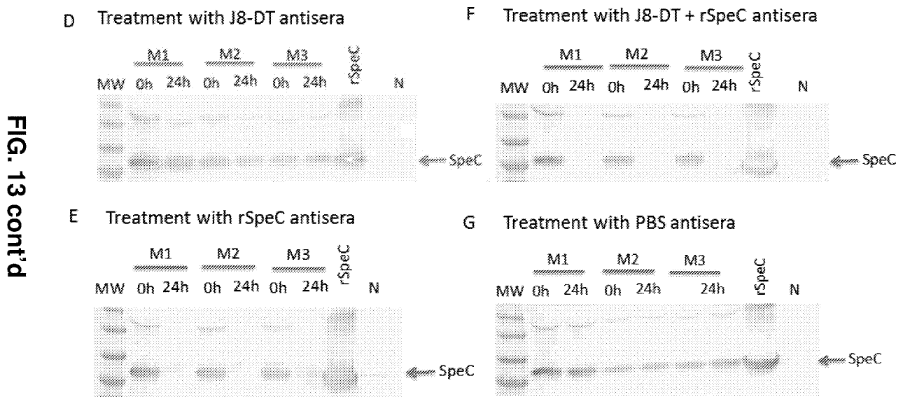

A further group of HLA-B6 mice were infected intraperitoneally with 1×10⁶ SN1 bacteria. These mice became ill more quickly and at 18 h post infection, when their average clinical scores were 10 [19], they were given 200μL of SpeC antisera, 200 μL of anti-J8 antisera, a combination of both, or 200μL of naïve serum, intravenously (FIG. 13A). All mice that received J8-DT and/or rSpeC antisera recovered within 24 h with significant reduction in clinical scores (P<0.01-P<0.001; FIG. 13B); however, it was only in those mice that received anti-J8 antibodies (either alone or in combination with anti-SpeC antibodies) that we observed bacterial clearance from blood and spleen (P<0.01; FIG. 13C), and only in those mice that received anti-rSpeC antibodies (either alone or in combination with anti-J8 antibodies) that we observed clearance of SpeC in the blood (using the WB assay) (FIG. 13D-G).

M-Protein from SN1 Exerts a Mitogenic Effect and Contributes to the Pro-Inflammatory Response

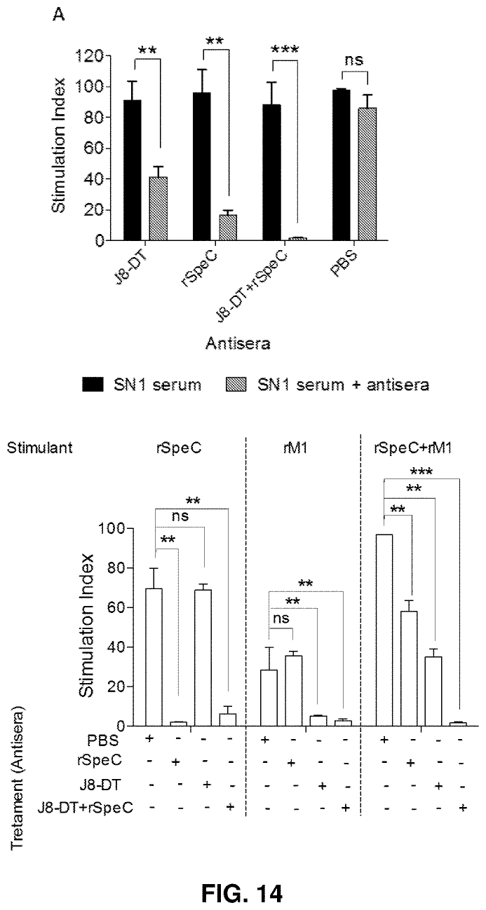

The ability of J8-DT and SpeC-specific antisera to treat STSS-like pathology in vivo, was further elucidated by in vitro studies. The mitogenic effect of serum from SN1-infected mice on HLA-B6 splenocytes was partially blocked by anti-SpeC and anti-J8-DT anti-serum but completely blocked by the combination of both antisera (FIG. 14B), indicating that J8-specific antibodies have a dual role in treating STSS in this model: they clear bacteria but also block the mitogenic effect of the emm89 M protein. This has a synergistic effect with the anti-SpeC serum. While unlikely, SN1 serum may contain other mitogenic factors that contain a J8-cross-reactive epitope. We thus asked whether anti-J8 antibodies would block the mitogenic effect of recM1. FIG. 14C shows that both recM1 and SpeC have mitogenic activity (as previously shown) and that the effect of both is additive. Furthermore, anti-J8-DT antisera blocks the mitogenic effect of recM1 completely. A combination of anti-J8-DT and anti-SpeC completely block the combined mitogenic activities of M1+SpeC. These data collectively indicate that anti-J8 antibodies can block the mitogenic activities of two distinct M proteins. The data do not suggest that the J8 epitope has mitogenic activity, simply that antibodies to J8 can neutralize the M protein. Others have suggested that the mitogenic determinant on the M protein is located in the aminoterminal half of the protein

Discussion:

The data presented here show that in a HLA-humanized mouse model it is possible to prevent STSS-like disease by vaccination and to rapidly treat established disease by specific immunotherapy containing antibodies to J8 and to SpeC. Antibodies to J8 have a dual effect: they eliminate the bacteria but also directly block the mitogenic effect of the M protein, while antibodies to SpeC block the activity of that protein. Together, the effect is synergistic and can completely resolve STSS-like disease.

Efforts to develop vaccines to prevent STSS are limited. One group has developed toxoids to SpeA and SpeC and shown that vaccination of rabbits can lead to antibodies that neutralize the toxin and protect rabbits from native toxin administered via a mini-osmotic pump. The rabbits were not exposed to a streptococcal infection [8, 9]. While this vaccine approach is promising, it suffers from the need to vaccinate with multiple toxoids to protect against only one aspect of streptococcal disease. Our data would suggest that this approach would not reduce bacterial sepsis. HLA transgenic mice have been used to show that certain HLA types are more prone to STTS [20], but not to model vaccine or therapy development for streptococcal STSS; however, they have been used to develop a candidate vaccine using defined non-toxic fragments of superantigens from Staphylococcus aureus [21]. These mice were not challenged with the organism, but with recombinant SAg.

We developed a candidate StrepA vaccine based on a highly conserved segment of the M protein (reviewed in [22]). The antigen is known as J8 and its sequence copies 12 amino acids of the C3-repeat of the M protein. Vaccination with J8 coupled to diphtheria toxoid (J8-DT) induces antibodies that opsonize StrepA in vitro, irrespective of the M-type, and can reduce bacterial burden following challenge and so protect mice from intraperitoneal, skin and mucosal challenge [14, 16, 23-25]. It was assumed that such vaccine-mediated protection would extend to protection against STSS, although this had not been tested in HLA-humanized mice. However, it was not assumed that passive immunotherapy with anti-J8 antibodies would resolve established disease, even if there was some reduction in bacterial burden since sAgs are believed to play a central role in the disease and there was no suggestion that antibodies to J8 would affect the levels of serum sAgs. We were surprised that 200 μL of J8-immune serum (with or without anti-SpeC antiserum) could virtually eliminate all the bacterial burden in the blood and spleen as well as resolved the clinical scores. Our data do not argue against an important role for SpeC or sAgs in the pathogenesis of STSS, particularly since anti-SpeC antibodies can also rapidly resolve clinical signs. However, they do argue that disease manifestations require more than sAg alone.

In addition to the SAgs, streptococcal M-protein has been reported to be associated with pro-inflammatory responses leading to severe streptococcal infections [26-29]. By stimulating monocytes via TLR2, the M-protein is capable of producing high amounts of pro-inflammatory cytokines. By working in synergy with neutrophil derived heparin binding protein (HBP), the M protein induces vascular leakage and contributes to pathophysiological consequences seen in severe streptococcal infections [30]. Some M proteins such as M1, M3 and M5 are consistently associated with outbreaks of ISD and STSS [31-33]. The B repeat region of M protein in certain serotypes such as M1 and M5 may also act as a superantigen and contribute towards inflammatory responses [34]. Although certain streptococcal serotypes (which distinguish strains with different surface M proteins) have been reported to be associated with ISD, it is thought that this association simply reflects the most common serotypes in the general population at that time [2]. Nevertheless, the M protein can down-regulate both innate and acquired immunity and may contribute to the pathogenesis of ISD.

The association of emm89 StrepA and SpeC with ISD has been noted in a number of recent reports and Japan where emm89 was the 2nd most predominant genotype found in STSS cases [35].

It is known that antibodies to the surface M protein (and to SAgs) are significantly lower in individuals who develop invasive disease [36] and it was suggested that the low levels of antibodies in the general population have contributed to the epidemic of ISD that started in the 1980s [37, 38]. However, it is not possible to determine whether the antibodies are low in individuals prior to infection or whether they become low after the infection commenced as a result of antibody catabolism. A direct way to address this issue is with an STSS model in which animals can be vaccinated and challenged or infected and treated.

We found that the presence of toxin was independent of systemic infection. SpeC was detected in the blood of mice following superficial skin infection without detectable bacteraemia. These mice did demonstrate pathological signs of disease. This is observed in some cases of clinical disease [39]. We noted that following superficial skin infection the toxin was detected in the infected serum at day 6 post-infection. This suggested a slow release of toxin during the initial phase of infection. This observation is in line with the Teflon tissue chamber model where expression of high levels of SpeA was noted on day 7 post-infection [40]. The acute onset of STSS flowing IP infection was quite apparent with toxin being detected in the blood of infected mice within 24 h post infection leading to high clinical scores. In contrast, superficial skin infection represented progressive onset of infection.

We demonstrated both in mice and humans, the typical pathology associated with mitogenic activity of SAgs. The amount of SpeC in the serum from infected mice had the potential to stimulate splenocytes from HLA-B6 mice to a level that was comparable (if not higher) to that caused by ConA or rSpeC. The proliferation caused by SN1 infected sera was higher than proliferation caused by rSpeC alone; thus suggesting the involvement of some other mitognic factors present in SN1 infected sera.

The addition of anti-SpeC antisera to the serum from infected mice was able to significantly inhibit the proliferative response, thus confirming that proliferation was largely due to SpeC. Nevertheless, the residual proliferation observed in treated group indicated the involvement of other virulence factors of StrepA including other SAg or M protein.

We noted that vaccination of HLA-B6 mice was efficacious in STSS prevention. Notably the mechanism of protection involved clearance of Strep A and not specific neutralisation of secreted SpeC. Vaccination with J8-DT resulted in significant reduction (>90%) in both local and systemic bacterial burden and thus protected mice from STSS related pathology. Furthermore, sera from vaccinated-infected mice were also shown to cause minimal proliferation of PBMC from healthy individuals. We believe that this effect could be attributed to lack of SpeC but also to lack of other factors in serum that are usually present as a result of StrepA infection and contribute towards overall disease outcome.

Passive immunotherapy holds promise as a means to treat STSS. Intravenous immunoglobulin (IVIG) has been shown to significantly reduce the case fatality of STSS [41]. This study used historical controls but in a more recent Swedish study of 67 patients with prospective controls, the mortality was 22 from 44 patients treated with antibiotics alone (50%) vs 3 from 23 (13%) in the group treated with IVIG plus antibiotics (P<0.01) [42]. However, it has been estimated that superantigen antibody titres of >40 in the IVIG are required for clinical benefit. This is approximately the amount of specific antibody that is found in IVIG and as such multiple doses of IVIG are recommended. The high costs of IVIG, batch-to-batch variation [43] and difficulties in supply underscore the need for alternative adjunctive therapies. A vaccine that prevented infection with all strains of StrepA or a specific antibody immunotherapy given at the time of diagnosis with or without antibiotics would have far greater utility. We found that administration of rSpeC antisera was able to neutralise the toxin, however it was unable to reduce bacterial burden in SN1 infected HLA-B6 mice. This observation underlined the fact that in order to treat an individual, multiple doses of anti rSpeC serum will be required until a complete clearance of toxin from the system is assured. However, as long as that individual harbours live StrepA, the concerns regarding toxins and related pathology will not be eliminated.

We hypothesized that a combination immunotherapy that would result in toxin neutralisation as well as clearance of StrepA from the system might be a better alternative. Clearance of Strep A will not only reduce the need for continual treatment for toxin neutralisation, but will also eliminate the possibility of other virulence factors contributing towards STSS pathology. In agreement with the previous reports we demonstrate that in HLA-B6 model StrepA SAgs may not be the sole determinant of the pathophysiology of STSS and other virulence factors of StrepA including the M-protein may play a critical role. By utilising emm89 Strep A isolate we were able to show that SAg SpeC and StrepA M protein work in alliance and contribute to the clinical disease as seen post-infection. In vivo neutralisation of M protein by J8-DT antisera prevents its interaction with fibrinogen and subsequent recognition via B2 integrins on neutrophils. As an end result, there is no activation and release of mediators of vascular leakage, which are a key occurrence in STSS. It is likely that in vitro neutralisation of M protein may have followed a different mechanism involving lack of cytokine induction and henceforth inflammatory responses.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, patent and scientific literature referred to herein is incorporated herein by reference in their entirety.

### REFERENCES

1. Lamagni, T. L., et al., Epidemiology of severe Streptococcus pyogenes disease in Europe. J Clin Microbiol, 2008. 46(7): p. 2359-67.
2. Steer, A. C., et al., Invasive group a streptococcal disease: epidemiology, pathogenesis and management. Drugs, 2012. 72(9): p. 1213-27.
3. Nelson, G. E., et al., Epidemiology of Invasive Group A Streptococcal Infections in the United States, 2005-2012. Clin Infect Dis, 2016. 63(4): p. 478-86.
4. Tyrrell, G. J., et al., Increasing Rates of Invasive Group A Streptococcal Disease in Alberta, Canada; 2003-2017. Open Forum Infect Dis, 2018. 5(8): p. ofy177.
5. Lamagni, T. L., et al., Severe Streptococcus pyogenes infections, United Kingdom, 2003-2004. Emerg Infect Dis, 2008. 14(2): p. 202-9.
6. Lamagni, E. a., Epidemiology of Streptococcus pyogenes”. In: Streptococcus Pyogenes. Basic biology to Clinical Manifestations. Eds: J J Ferretti, D L Stevens, V A Fischetti. 2017. University of Oklahoma Health Sciences Center, Oklahoma. https://http://www.ncbi.nlm.nih.gov/books/NBK343616), 2017: p. pp 601-627.
7. Fraser, P. a., Streptococcal superantigens: Biological properties and potential role in disease”, In: Streptococcus Pyogenes. Basic biology to Clinical Manifestations. Eds: J J Ferretti, D L Stevens, V A Fischetti. 2017. University of Oklahoma Health Sciences Center, Oklahoma. https://http://www.ncbi.nlm.nih.gov/books/NBK343616). 2017: p. pp 445-485.
8. Roggiani, M., et al., Toxoids of streptococcal pyrogenic exotoxin A are protective in rabbit models of streptococcal toxic shock syndrome. Infect Immun, 2000. 68(9): p. 5011-7.
9. McCormick, J. K., et al., Development of streptococcal pyrogenic exotoxin C vaccine toxoids that are protective in the rabbit model of toxic shock syndrome. J Immunol, 2000. 165(4): p. 2306-12.
10. Hackett S. P., S. D. L., Streptococcal toxic shock syndrome: synthesis of tumor necrosis factor and interleukin-1 by monocytes stimulated with pyrogenic exotoxin A and streptolysin O. The Journal of Infectious Diseases., 1992. 165(5): p. 879-885.
11. Hackett, S., Ferretti, J. J., & Stevens, D. L., Cytokine induction by viable group A streptococci: suppression by streptolysin O. The 94th Annual Meeting of the American Society for Microbiology 1994. 73.
12. Muller-Alouf, H., et al., Comparative study of cytokine release by human peripheral blood mononuclear cells stimulated with Streptococcus pyogenes superantigenic erythrogenic toxins, heat-killed streptococci, and lipopolysaccharide. Infect Immun, 1994. 62(11): p. 4915-21.
13. Kotb, M., et al., Temporal relationship of cytokine release by peripheral blood mononuclear cells stimulated by the streptococcal superantigen pep M5. Infect Immun, 1993. 61(4): p. 1194-201.
14. Batzloff, M. R., et al., Protection against group A Streptococcus by immunization with J8-diphtheria toxoid: contribution of J8- and diphtheria toxoid-specific antibodies to protection. J Infect Dis, 2003. 187(10): p. 1598-608.
15. Pandey, M., M. R. Batzloff, and M. F. Good, Mechanism of protection induced by group A Streptococcus vaccine candidate J8-DT: contribution of B and T-cells towards protection. PLoS One, 2009. 4(4): p. e5147.
16. Pandey, M., et al., A synthetic M protein peptide synergizes with a CXC chemokine protease to induce vaccine-mediated protection against virulent streptococcal pyoderma and bacteremia. J Immunol, 2015. 194(12): p. 5915-25.
17. Caro-Aguilar, I., et al., Immunogenicity in mice and non-human primates of the Group A Streptococcal J8 peptide vaccine candidate conjugated to CRM197. Hum Vaccin Immunother, 2013. 9(3): p. 488-96.
18. Silvana Sekuloskil ¶, M. B., Paul Griffin1,2,3,4, William Parsonage4, Suzanne Elliott2, Jon Hartas5, Peter O'Rourke1, Manisha Pandey5, Tania . . . Fran Rubin6, Robin Mason6, Jonathan Carapetis7, James McCarthy1,4*¶ and Michael F Good5*¶, Evaluation of safety and immunogenicity of a group A Streptococcus vaccine candidate (MJ8VAX) in a randomized clinical trial PLoSOne, 2018.
19. Shrum, B., et al., A robust scoring system to evaluate sepsis severity in an animal model. BMC Res Notes, 2014. 7: p. 233.
20. Nooh, M. M., et al., HLA transgenic mice provide evidence for a direct and dominant role of HLA class II variation in modulating the severity of streptococcal sepsis. J Immunol, 2007. 178(5): p. 3076-83.
21. DaSilva, L., et al., Humanlike immune response of human leukocyte antigen-DR3 transgenic mice to staphylococcal enterotoxins: a novel model for superantigen vaccines. J Infect Dis, 2002. 185(12): p. 1754-60.
22. Good, M. F., et al., Strategic development of the conserved region of the M protein and other candidates as vaccines to prevent infection with group A streptococci. Expert Rev Vaccines, 2015. 14(11): p. 1459-70.
23. Pandey, M., et al., Combinatorial Synthetic Peptide Vaccine Strategy Protects against Hypervirulent CovR/S Mutant Streptococci. J Immunol, 2016. 196(8): p. 3364-74.
24. Pandey, M., et al., Physicochemical characterisation, immunogenicity and protective efficacy of a lead streptococcal vaccine: progress towards Phase I trial. Sci Rep, 2017. 7(1): p. 13786.
25. Zaman, M., et al., Novel platform technology for modular mucosal vaccine that protects against Streptococcus. Sci Rep, 2016. 6: p. 39274.
26. Tomai, M. A., P. M. Schlievert, and M. Kotb, Distinct T-cell receptor V beta gene usage by human T lymphocytes stimulated with the streptococcal pyrogenic exotoxins and pep M5 protein. Infect Immun, 1992. 60(2): p. 701-5.
27. Watanabe-Ohnishi, R., et al., Characterization of unique human TCR V beta specificities for a family of streptococcal superantigens represented by rheumatogenic serotypes of M protein. J Immunol, 1994. 152(4): p. 2066-73.
28. Perez-Lorenzo, R., et al., Peripheral blood mononuclear cells proliferation and Th1/Th2 cytokine production in response to streptococcal M protein in psoriatic patients. Int J Dermatol, 2006. 45(5): p. 547-53.
29. Pahlman, L. I., et al., Streptococcal M protein: a multipotent and powerful inducer of inflammation. J Immunol, 2006. 177(2): p. 1221-8.
30. Herwald, H., et al., M protein, a classical bacterial virulence determinant, forms complexes with fibrinogen that induce vascular leakage. Cell, 2004. 116(3): p. 367-79.
31. Bisno, A. L., Group A streptococcal infections and acute rheumatic fever. N Engl J Med, 1991. 325(11): p. 783-93.
32. Robinson, J. H. and M. A. Kehoe, Group A streptococcal M proteins: virulence factors and protective antigens. Immunol Today, 1992. 13(9): p. 362-7.
33. Meisal, R., et al., Sequence type and emm type diversity in Streptococcus pyogenes isolates causing invasive disease in Norway between 1988 and 2003. J Clin Microbiol, 2008. 46(6): p. 2102-5.
34. Wang, B., et al., Localization of an immunologically functional region of the streptococcal superantigen pepsin-extracted fragment of type 5 M protein. J Immunol, 1993. 151(3): p. 1419-29.
35. Darenberg, J., et al., Molecular and clinical characteristics of invasive group A streptococcal infection in Sweden. Clin Infect Dis, 2007. 45(4): p. 450-8.
36. Basma, H., et al., Risk factors in the pathogenesis of invasive group A streptococcal infections: role of protective humoral immunity. Infect Immun, 1999. 67(4): p. 1871-7.
37. Holm, S. E., et al., Aspects of pathogenesis of serious group A streptococcal infections in Sweden, 1988-1989. J Infect Dis, 1992. 166(1): p. 31-7.
38. Stevens, D. L., Invasive group A Streptococcus infections. Clin Infect Dis, 1992. 14(1): p. 2-11.
39. Darenberg, J., et al., Intravenous immunoglobulin G therapy in streptococcal toxic shock syndrome: a European randomized, double-blind, placebo-controlled trial. Clin Infect Dis, 2003. 37(3): p. 333-40.
40. Kazmi, S. U., et al., Reciprocal, temporal expression of SpeA and SpeB by invasive MilT group a streptococcal isolates in vivo. Infect Immun, 2001. 69(8): p. 4988-95.
41. Kaul, R., et al., Intravenous immunoglobulin therapy for streptococcal toxic shock syndrome—a comparative observational study. The Canadian Streptococcal Study Group. Clin Infect Dis, 1999. 28(4): p. 800-7.
42. Linner, A., et al., Clinical efficacy of polyspecific intravenous immunoglobulin therapy in patients with streptococcal toxic shock syndrome: a comparative observational study. Clin Infect Dis, 2014. 59(6): p. 851-7.
43. Jolles, S., W. A. Sewell, and S. A. Misbah, Clinical uses of intravenous immunoglobulin. Clin Exp Immunol, 2005. 142(1): p. 1-11.

1. (canceled)

2. A method of passively immunizing a mammal against an invasive group A Streptococcus disease (iGAS), such as streptococcal toxic shock syndrome, said method comprising the step of administering to the mammal an antibody or antibody fragment that binds, or is raised against, a group A Streptococcus M protein fragment, or a variant or derivative thereof, wherein the M protein fragment is or comprises a conserved region of the M protein; and optionally an antibody or antibody fragment that binds, or is raised against, a group A Streptococcus superantigen protein, fragment, variant or derivative thereof; to thereby passively immunize the mammal against the iGAS in the mammal.

3. A method of treating or preventing an invasive group A Streptococcus disease (iGAS), such as streptococcal toxic shock syndrome in a mammal, said method comprising the step of administering to the mammal an antibody or antibody fragment that binds, or is raised against, a group A Streptococcus M protein fragment, or a variant or derivative thereof, wherein the M protein fragment is or comprises a conserved region of the M protein; and optionally an antibody or antibody fragment that binds, or is raised against, a group A Streptococcus superantigen protein, fragment, variant or derivative thereof; to thereby treat or prevent the iGAS in the mammal.

4. A composition formulated for administration to a mammal, said composition comprising:
an antibody or antibody fragment that binds, or is raised against, a group A Streptococcus M protein fragment, or a variant or derivative thereof, wherein the M protein fragment is or comprises a conserved region of the M protein; and
an antibody or antibody fragment that binds, or is raised against, a group A Streptococcus superantigen protein, fragment, variant or derivative thereof.

5. The method of claim 2, wherein the M protein fragment is selected from the group consisting of an M protein fragment that is, comprises, or is contained within a p145 peptide; an M protein fragment that is, comprises, or is contained within a J8 peptide, or a fragment, variant or derivative thereof; and an M protein fragment that is, comprises, or is contained within a p17 peptide, or a fragment, variant or derivative thereof; wherein the M protein fragment is, comprises, or is contained within an amino acid sequence selected from the group consisting of SEQ ID NOs:1-10 and 13 to 29.

6-8. (canceled)

9. The method of claim 2, wherein the superantigen is streptococcal pyrogenic exotoxin (Spe) A or SpeC.

10. The method of claim 2, wherein the antibody or antibody fragment that binds, or is raised against, a group A Streptococcus M protein, fragment, variant or derivative thereof and/or the antibody or antibody fragment that binds, or is raised against, a group A Streptococcus superantigen protein, fragment, variant or derivative thereof is a monoclonal antibody or antibody fragment.

11. The method of claim 2, wherein the antibody or antibody fragment that binds, or is raised against, a group A Streptococcus M protein, fragment, variant or derivative thereof and/or the antibody or antibody fragment that binds, or is raised against, a group A Streptococcus superantigen protein, fragment, variant or derivative is a humanized monoclonal antibody or antibody fragment.

12. (canceled)

13. The method of claim 2, wherein the iGAS is streptococcal toxic shock syndrome (STSS).

14. The method of claim 2, wherein said method comprises administering both the antibody or antibody fragment that binds, or is raised against, a group A Streptococcus M protein fragment, or a variant or derivative thereof, and the antibody or antibody fragment that binds, or is raised against, a group A Streptococcus superantigen protein, fragment, variant or derivative thereof.

15. The method of claim 3, wherein the M protein fragment is selected from the group consisting of an M protein fragment that is, comprises, or is contained within a p145 peptide; an M protein fragment that is, comprises, or is contained within a J8 peptide, or a fragment, variant or derivative thereof; and an M protein fragment that is, comprises, or is contained within a p17 peptide, or a fragment, variant or derivative thereof, wherein the M protein fragment is, comprises, or is contained within an amino acid sequence selected from the group consisting of SEQ ID NOs:1-10 and 13 to 29.

16. The method of claim 3, wherein the superantigen is streptococcal pyrogenic exotoxin (Spe) A or SpeC.

17. The method of claim 3, wherein the antibody or antibody fragment that binds, or is raised against, a group A Streptococcus M protein, fragment, variant or derivative thereof, and/or the antibody or antibody fragment that binds, or is raised against, a group A Streptococcus superantigen protein, fragment, variant or derivative thereof is a monoclonal antibody or antibody fragment.

18. The method of claim 3, wherein the antibody or antibody fragment that binds, or is raised against, a group A Streptococcus M protein fragment, or a variant or derivative thereof; and/or the antibody or antibody fragment that binds, or is raised against, a group A Streptococcus superantigen protein, fragment, variant or derivative is a humanized monoclonal antibody or antibody fragment.

19. The method of claim 3, wherein said method comprises administering both the antibody or antibody fragment that binds, or is raised against, a group A Streptococcus M protein fragment, or a variant or derivative thereof, and the antibody or antibody fragment that binds, or is raised against, a group A Streptococcus superantigen protein, fragment, variant or derivative thereof.

20. The method of claim 3, wherein the iGAS is streptococcal toxic shock syndrome (STSS).

21. The composition of claim 4, wherein the M protein fragment is selected from the group consisting of an M protein fragment that is, comprises, or is contained within a p145 peptide; an M protein fragment that is, comprises, or is contained within a J8 peptide, or a fragment, variant or derivative thereof; an M protein fragment that is, comprises, or is contained within a p17 peptide, fragment, variant or derivative thereof, wherein the M protein fragment is, comprises, or is contained within an amino acid sequence selected from the group consisting of SEQ ID NOs:1-10 and 13 to 29.

22. The composition of claim 4, wherein the superantigen is streptococcal pyrogenic exotoxin (Spe) A or SpeC.

23. The composition of claim 4, wherein the antibody or antibody fragment that binds, or is raised against, a group A Streptococcus M protein, fragment, variant or derivative thereof; and/or the antibody or antibody fragment that binds, or is raised against, a group A Streptococcus superantigen protein, fragment, variant or derivative thereof is a monoclonal antibody or antibody fragment.

24. The composition of claim 4, wherein the antibody or antibody fragment that binds, or is raised against, a group A Streptococcus M protein, fragment, variant or derivative thereof; and/or the antibody or antibody fragment that binds, or is raised against, a group A Streptococcus superantigen protein, fragment, variant or derivative is a humanized monoclonal antibody or antibody fragment.

25. The method of claim 2, wherein the mammal is a human.
